(19) United States
(12) Patent Application Publication
(10) Pub. No.: US 2023/0279471 A1
(43) Pub. Date: Sep. 7, 2023

(54) METHODS FOR SEPARATING AND DETECTING DOUBLE-STRANDED AND SINGLE-STRANDED RIBONUCLEIC ACID (RNA)

(71) Applicant: PerkinElmer Health Sciences, Inc., Waltham, MA (US)

(72) Inventors: Lloyd Bwanali, Westborough, MA (US); Guangnan Meng, Sudbury, MA (US); Shreyas Shah, Shrewsbury, MA (US)

(21) Appl. No.: 18/115,405

(22) Filed: Feb. 28, 2023

Related U.S. Application Data
(60) Provisional application No. 63/314,936, filed on Feb. 28, 2022.

(51) Int. Cl. C12Q 1/6809; G01N 33/58

(52) U.S. Cl. CPC C12Q 1/6809; G01N 33/582

(57) ABSTRACT

Provided herein are capillary electrophoresis methods for separating and detecting double-stranded ribonucleic acid (dsRNA) contaminants in samples of single-stranded ribonucleic acids (ssRNA) such as RNA therapies (e.g., mRNA vaccines).

### CLAIM OF PRIORITY

This application claims the benefit of U.S. Provisional Patent Application No. 63/314,936, filed on Feb. 28, 2022, which is incorporated by reference herein in its entirety.

### BACKGROUND

Single-stranded RNA (ssRNA) is a key component in many RNA therapies such as messenger (mRNA) vaccines. However, double-stranded RNA (dsRNA) is a byproduct of manufacturing of ssRNA therapies and it is a major trigger of unwanted immune responses. It is therefore of interest to develop new assays for detecting dsRNA contaminants in ssRNA therapies to ensure their safety and efficacy.

### SUMMARY OF THE INVENTION

Provided herein are methods for separating and detecting dsRNA and ssRNA in a high-throughput manner that is rapid and reproducible. Such methods involve separating dsRNA and ssRNA via capillary electrophoresis and then detecting the separated dsRNA and ssRNA via a signal produced by an intercalating agent bound to dsRNA and ssRNA.

Accordingly, aspects of the present disclosure provide a method for separating and detecting double-stranded ribonucleic acids (dsRNAs) from single-stranded ribonucleic acids (ssRNAs) by capillary electrophoresis, the method comprising introducing a polymer solution and a sample comprising dsRNAs and ssRNAs into a first capillary channel, wherein the polymer solution is introduced prior to the sample, and wherein the polymer solution or the sample comprises an intercalating agent; injecting a portion of the sample into a second capillary channel that intersects and is fluidly connected with the first capillary channel; applying a voltage gradient across the length of the second capillary channel to separate dsRNAs into a first segment of the portion of the sample and ssRNAs into a second segment of the portion of the sample; and detecting an amount of intercalating agent in the first segment and the second segment, wherein the amount of intercalating agent in the first segment is indicative of an amount of dsRNA in the sample and the amount of intercalating agent in the second segment is indicative of an amount of ssRNA in the sample.

In some embodiments, the intercalating agent is fluorescent and detecting the amount of intercalating agent comprises detecting a fluorescent signal. In some embodiments, the concentration of the intercalating agent in the polymer solution or in the sample is between 0.01 mM and 0.2 mM. In some embodiments, the intercalating agent is selected from the group consisting of SYBR dyes, ethidium bromide, acridine orange, propidium iodide, 7-aminoactinomycin D (7-AAD), 4′,6-diamidino-2-phenylindole (DAPI), cyanine dyes, Alexa Fluor dyes, BODIPY dyes, and combinations thereof.

In some embodiments, the concentration of the polymer in the polymer solution is between 0.01% to 20% (w/v). In some embodiments, the polymer has a molecular weight between 1 Kd to 5,000 Kd.

In some embodiments, the polymer is formed from a monomer selected from the group consisting of acrylic acid, carboxylic acid, sulfonic acid, phosphoric acid monomer, bisacrylamidoacetic acid, 4,4-Bis(4-hydroxyphenyl)pentanoic acid, 3-butene-1,2,3-tricarboxylic acid, 2-carboxyethylacrylate, itaconic acid, methacrylic acid, and 4-vinylbenzoic acid.

In some embodiments, the polymer is an acrylic polymer. In some embodiments, the acrylic polymer is selected from the group consisting of a polyacrylamide polymer, a polymethylacrylamide polymer, a polydimethylacrylamide polymer, and a polydimethylacrylamide-co-acrylic acid polymer.

In some embodiments, the polymer comprises a net charge of between 0.01% and 2% and the net charge is the same charge as at least one surface of the capillary channel.

In some embodiments, the polymer comprises negatively charged monomer subunits and the at least one surface of the capillary channel has a negative surface charge. In some embodiments, the negatively charged monomer subunits are selected from the group consisting of are selected from acrylic acid, bisacrylamidoacetic acid, 4,4-Bis(4-hydroxyphenyl)pentanoic acid, 3-butene-1,2,3-tricarboxylic acid, 2-carboxyethylacrylate, itaconic acid, methacrylic acid, 4-vinylbenzoic acid, sulfonic acid, 2-acrylamido-2-methyl-1-propanesulfonic acid, 2-methyl-2-propene-1-sulfonic acid, 2-propene-1-sulfonic acid, 4-styrenesulfonic acid, 2-sulfoethyl methacrylate, 3-sulfopropyldimethyl-3-methacrylamidopropylammonium inner salt, 3-sulfopropyl methacrylate, vinylsulfonic acid, Bis(2-methacryloxyethyl)phosphate, and monoacryloxyethyl phosphate.

In some embodiments, the polymer comprises positively charged monomer subunits and the at least one surface of the capillary channel has a positive surface charge. In some embodiments, the positively charged monomer subunits are selected from the group consisting of 2-acryloxyethyltrimethylammonium chloride, diallyldimethylammonium chloride, 2-methacryloxyethyltrimethylammonium chloride, and 3-methacryloxy-2-hydroxypropyltrimethylammonium chloride.

In some embodiments, the concentration of dsRNA in the sample is between 1 pg/μL and 100 pg/μL. In some embodiments, the concentration of ssRNA in the sample is between 1 pg/μL and 100 pg/μL.

In some embodiments, injecting the portion of the sample from the capillary channel into the second capillary channel comprises applying a voltage difference through the capillary channel to electrokinetically move the portion of the sample from the capillary channel into the second capillary channel.

In some embodiments, methods described herein further comprise introducing an additional polymer solution and the sample into the first capillary channel, wherein the additional polymer solution is introduced prior to the sample, and wherein the additional polymer solution or the sample comprises an additional intercalating agent; injecting a portion of the sample into the second capillary channel that intersects and is fluidly connected with the first capillary channel; applying a second voltage gradient across the length of the second capillary channel to separate dsRNAs into an additional first segment of the portion of the sample and ssRNAs into an additional second segment of the portion of the sample; and detecting an amount of additional intercalating agent in the additional first segment and the additional second segment, wherein the amount of additional intercalating agent in the additional first segment is indicative of an amount of dsRNA in the sample and the amount of additional intercalating agent in the additional second segment is indicative of an amount of ssRNA in the sample.

In some embodiments, one or more steps are performed in a microfluidic device.

Aspects of the present disclosure provide a method for separating and detecting double-stranded ribonucleic acids (dsRNAs) from single-stranded ribonucleic acids (ssRNAs) in a microfluidic device, the method comprising providing a microfluidic device comprising a substrate having a surface; an analysis channel disposed in the substrate; a sample loading channel disposed in the substrate and intersecting the analysis channel at an intersection; and introducing a polymer solution and a sample comprising dsRNAs and ssRNAs into the loading channel, wherein the polymer solution is introduced prior to the sample, and wherein the polymer solution or the sample comprises an intercalating agent; injecting a portion of the sample into the analysis channel; applying a voltage gradient across the length of the analysis channel to separate dsRNAs into a first segment of the portion of the sample and ssRNAs into a second segment of the portion of the sample; and detecting an amount of intercalating agent in the first segment and the second segment, wherein the amount of intercalating agent in the first segment is indicative of an amount of dsRNA in the sample and the amount of intercalating agent in the second segment is indicative of an amount of ssRNA in the sample.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIGS. 1A-1E are schematic illustrations showing sample loading and injection in accordance with some embodiments of the technology described herein.

FIGS. 2A-2F are schematic illustrations showing separation of dsRNA and ssRNA into portions of the sample and detection of the separated dsRNA and ssRNA in accordance with some embodiments of the technology described herein.

FIG. 3 is a schematic illustrating reagent placement on the chip.

FIG. 4 is a graph showing the chromatographic separation of the dsDNA standard sample in accordance with some embodiments of the technology described herein. The inset is an image showing separation of the dsDNA standard sample by agarose gel electrophoresis.

FIG. 5 is a graph showing the chromatographic separation of the ssDNA standard sample in accordance with some embodiments of the technology described herein. The inset is an image showing separation of the ssDNA standard sample by agarose gel electrophoresis.

FIG. 6 is an overlay of chromatographs from the chromatographic separation of dsDNA and ssDNA in FIG. 4 and FIG. 5, respectively.

FIG. 7 is graphs showing the chromatographic separation of the dsRNA standard sample using a standard pinch flow (top panel) and an improved pinch flow (bottom panel) in accordance with some embodiments of the technology described herein.

FIG. 8 is a schematic depiction of a sample preparation and analysis workflow for detecting dsRNA contaminants in mRNA samples (e.g., mRNA vaccines) in accordance with some embodiments of the technology described herein.

FIGS. 9A-9B are graphs showing the chromatographic separation of the dsRNA ladder (FIG. 9A) and the ssRNA ladder (FIG. 9B) with heating (dashed line) and without heating (solid line) during sample preparation. Experiments were performed using a 6% gel matrix.

FIGS. 10A-10B are graphs showing fluorescence detection of dsRNA (FIG. 10A) and ssRNA (FIG. 10B) using RNA dye (triangle) and DNA dye (square) at the indicated concentrations.

FIG. 11 is an electropherogram showing overlay of a ssRNA ladder analyzed using 6% gel matrix (dashed line) and then using a 3% gel matrix (solid line).

FIGS. 12A-12C are graphs showing migration differences between a dsRNA (solid line) and an ssRNA (dashed line) ladder using a DNA dye. Electropherogram (FIG. 12A) showing the overlay of both ladders, migration time (FIG. 12B) and mobility (FIG. 12C) over fragment size for each molecule type. Note that in the case of dsRNA the fragment sizes are in bp while for the ssRNA the fragment sizes are in nt.

FIGS. 13A-13F are graphs showing data from analysis of fluorescent labeling differences between a dsRNA and an ssRNA ladder using SYTO™ 61 and RiboRed. Electropherogram (FIG. 13A) showing the overlay of the dsRNA ladder labeled with both dye types, the average area (FIG. 13B) and peak height (FIG. 13C) produced by each fragment of the dsRNA ladder. Electropherogram (FIG. 13D) showing the overlay of the ssRNA ladder labeled with both dye types, the average area (FIG. 13E) and peak height (FIG. 13F) produced by each fragment of the ssRNA ladder. Note that in the case of dsRNA the fragment sizes are in bp while for the ssRNA the fragment sizes are in nt.

FIGS. 14A-14C are graphs showing data from analysis of samples including custom long dsRNA and long mRNA using SYTO™ 61. Electropherogram (FIG. 14A), summarized migration (FIG. 14B) and summarized mobility (FIG. 14C) of a 4,001 bp and nt, respectively, dsRNA and mRNA fragments using STYO™ 61. The summarized migration times includes data from three independent runs, each with two to three repeats.

FIGS. 15A-15F are graphs showing data from analysis of samples including custom long dsRNA and long mRNA using SYTO™ 61 and RiboRed. Custom dsRNA fragment electropherogram (FIG. 15A), area under the curve (FIG. 15B) and peak height (FIG. 15C) using SYTO™ 61 and RiboRed. Custom mRNA fragment electropherogram (FIG. 15D), area under the curve (FIG. 15E) and peak height (FIG. 15F) using SYTO™ 61 and RiboRed. The summarized data includes results from three independent runs, each with two to three repeats.

FIGS. 16A-16E are graphs showing data from analysis of mixed long dsRNA and mRNA samples. Electropherogram of mixed long dsRNA and mRNA samples where SYTO™ 61 is represented in a solid line, and RiboRed is represented in a dashed line (FIG. 16A). dsRNA area under the curve (FIG. 16B) and peak height (FIG. 16C). mRNA area under the curve (FIG. 16D) and peak height (FIG. 16E). The summarized data includes results from three independent runs, each with two to three repeats.

FIGS. 17A-17B are graphs showing ratios between the RNA and the DNA dye (RiboRed/SYTO™ 61) for dsRNA (black) and mRNA (gray) when analyzed individually (FIG. 17A) and mixed (FIG. 17B). These results include data from three independent runs, each with two to three repeats.

FIGS. 18A-18B are graphs showing assessment of the linearity in the response of dsRNA (FIG. 18A) and mRNA (FIG. 18B) as their concentrations increased using SYTO™ 61 and RiboRed. In FIG. 18A, the line of best fit for the dsRNA sample using SYTO™ 61 was y=79.05x+63.87 with an R²=0.93 while that of RiboRed was y=6.49x+39.21 with an R²=0.75. In FIG. 18B, the line of best fit for the mRNA sample using SYTO™ 61 was y=278.2x+1189 with an R²=0.84 while that of RiboRed was y=503.0x+1103 with an R²=0.75.

FIGS. 19A-19B are graphs showing assessment of dye ratios (RiboRed/SYTO™ 61) at varying concentrations of dsRNA. Dye ratios represented 5.0%, 7.5%, and 10.0% of the total mRNA concentration. FIG. 19A is a graph showing comparison between the dsRNA (black) and mRNA (gray) dye ratios at varying concentrations of dsRNA. FIG. 19B is a graph showing comparison among the dsRNA dye ratios and among mRNA dye ratios, separately, as the percentage of dsRNA relative to that of mRNA varied from 5.0% (black), to 7.5% (gray), to 10.0% (light gray).

The details of one or more embodiments of the invention are set forth in the description below. Other features or advantages of the present invention will be apparent from the following drawings and detailed description of several embodiments, and also from the appended claims.

### DETAILED DESCRIPTION OF THE INVENTION

The currently available methods for detecting dsRNA contaminants in ssRNA therapies utilize high performance liquid chromatography (HPLC). These methods are low-throughput and time consuming (>30 minutes per sample). It is therefore of interest to develop new assays for detecting dsRNA contaminants in ssRNA therapies to ensure their safety and efficacy.

The present disclosure is based, at least in part, on the development of capillary electrophoresis methods for separating and detecting dsRNA and ssRNA in a sample. Methods described herein can be used for detecting small amounts of dsRNA in a sample of ssRNA. As such, methods described herein are particularly useful in evaluating purity of ssRNA therapies such as mRNA therapies.

mRNA vaccines (e.g., COVID-19 vaccine) offer various advantages over traditional vaccines in preventing and reducing disease, as well as shortening the time between pathogen discovery and vaccine creation. Production of mRNA vaccines results in a number of nucleic acid and enzymatic by-products, most of which can be detected and removed; however, double stranded RNA (dsRNA) contaminants pose a particular challenge. Current purification and detection platforms for dsRNA vary in effectiveness, with problems in scalability for mass mRNA vaccine production. Effectively detecting dsRNA is crucial in ensuring the safety and efficacy of the vaccines as these strands can cause autoimmune reactions with length-symptom dependency and they can enhance mRNA degradation. Therefore, the studies described herein analyzed behavior differences in labeling of mRNA and dsRNA molecules in order to develop a high throughput microfluidic platform for dsRNA contaminant detection. The limit of detection of the system for dsRNA was determined to be between 17.7-76.6 pg/μL while that of mRNA was between 1.79-2.83 pg/μL, with a maximum loading capacity of mRNA of 12.99 ng/μL. Based on these values, the proposed method should allow for the detection of dsRNA contaminants that are present in percentages as low as 0.14-0.59% in comparison to the total mRNA concentration.

Vaccines have been a crucial public health measure in preventing and reducing diseases, morbidity, and mortality by millions each year [1]. Vaccine importance has been highlighted by the COVID-19 pandemic and the widespread use of newly developed mRNA vaccines. While the possibilities of mRNA vaccines have been noted since 1990, there have been many bottlenecks that have not allowed their mass production and distribution [2]. Nevertheless, much research has gone into nucleic acid-based vaccines due to their ability to provide precise targeting of the immune response and offer advantages in safety, efficiency, and specificity when compared to other vaccine platforms [3], [4]. mRNA vaccines, specifically, offer advantages including extranuclear activity, which confers a very low risk for subsequent random genome integration and insertional mutagenesis, more controlled expression of coded antigen, no inclusion of foreign genes, and the ability to be produced in a cell-free environment by in vitro transcription [3], [5]. If this technology is harnessed for widespread and large-scale use, mRNA vaccine application could extend to cancer therapies, therapeutic protein replacement therapies, treatment of genetic diseases, and a wide variety of infectious diseases [3], [5].

Challenges that mRNA vaccine production and manufacturing have faced include mRNA instability, in vivo delivery method, and innate immunogenicity. The instability of the mRNA has been mitigated with the optimization of untranslated regions [5], [6]. After testing various delivery methods, the use of lipid nanoparticles (LPNs) has been found to be most effective in protecting mRNA from rapid degradation by extracellular RNase and allowing the mRNA to travel to the target cell for cellular uptake and protein production [3], [7]. The body's recognition of exogenous mRNA can stimulate a robust immune response, which can negatively affect the desired immune response [4]. This latter problem has been resolved by introducing modified nucleosides such as pseudouridine and 1-methyl pseudouridine [4].

However, in addition to the aforementioned hurdles that have largely been addressed, due to the nature of the development process of in vitro transcribed (IVT) mRNA, there exists the possibility of contaminants such as leftover enzymes, free nucleotides, residual DNA, truncated RNA, and dsRNA [4]. These exogenous contaminants, specifically dsRNA, which has been identified as a major contaminant, are potent pathogen-associated molecular patterns (PAMPs). The creation of dsRNA in IVT production of mRNA stems from the activity of T7 RNA polymerase and two potential consequent scenarios: 1) 3′ extension of the run-off product that anneals to a complementary sequence in the body of run-off transcripts; and 2) hybridization of antisense RNA to the run-off transcript [8]. When recognized, PAMPs can cause type I interferon activity, which leads to the inhibition of translation and the enhancement of mRNA degradation. This, in turn, can result in decreased production of the desired antigen protein. dsRNA contaminants can also activate pro-inflammatory cytokines associated with potential autoimmune reactions, including effects to the central nervous system [4], [9]-[11]. Additionally, there appears to be a positive correlation between dsRNA fragment length and the associated negative effects [12], suggesting that careful monitoring of both presence and length of dsRNA fragments are required to ensure the safety of the vaccine.

Current mRNA vaccine purification platforms may include DNase digestion, precipitation, chromatography, or tangential flow filtration; however, there remains a lack of well-established manufacturing platforms for mRNA, so a number of mRNA synthesis and purification techniques could be combined [5]. The pharmaceutical industry often utilizes different forms of chromatography coupled with tangential flow filtration for purification due its versatility, cost-effectiveness, selectivity, and importantly, its ability to be upscaled for mass production of mRNA vaccines [5]. However, different forms of this technique come with their own limitations. For example, ion pair reverse phase chromatography is an excellent purification method that removes dsRNA while maintaining high yield, but it is very costly to scale and uses toxic reagents such as acetonitrile [5], [13]. Conversely, anion exchange chromatography can be used to cost-effectively purify mRNA at a large-scale, but it requires denaturing conditions, tight control of temperature, and use of potential chaotropic agents [5]. Lastly, high performance liquid chromatography has shown to remove dsRNA and other contaminants, resulting in a 10- to 1000- fold increase in protein production levels [14], but there are issues with both the scale-up process and mRNA stability [13]. New innovative purification techniques are being researched such as cellulose-based chromatography, which exploits the ability of dsRNA to bind to cellulose in the presence of ethanol. This process may offer a simple, efficient, high-throughput, and cost-effective alternative for the removal of dsDNA [15].

However, even with these various purification platforms, some having evidence of dsRNA removal of over 90% [15], the variability in quality and effectiveness of current purification methods calls for quality control systems for the detection of residual and harmful dsRNA fragments in mRNA vaccines. Current preferred quality control detection assays for characterizing RNA transcripts include UV spectroscopy, fluorescence-based assays, electrophoresis on gels, western blot for dsRNA, immunoassays (e.g., enzyme-linked immunosorbent assay), enzyme-linked immunosorbent assay, and chromatography. However, these are severely limited by factors such as resolution, precise handling, hazardous reagents, intense labor, need for antibodies, long run time, or inability to be scaled [13], [16]-[19], leaving an important need for fast, high throughput, low-cost quality control systems. This can be accomplished using microfluidics, whose versatility allows for the fast development of novel analytical methodologies.

With its great automation compatibility and low volume requirements, the development of microfluidics has revolutionized the way in which samples are analyzed. Broadly, microfluidics refers to methods or devices that process or manipulate microscale amounts (10⁻⁹ to 10⁻¹⁸ L) of fluids within systems that have dimensions in the scale of tens to hundreds of microns [20]. As a result of the miniaturization of traditional biomolecular analysis tools, the decrease in overall scales offers precise control of fluids, high-throughput capabilities, and rapid sample processing, make it capable of outperforming traditional technologies, often leading to lower-cost alternatives [20], [21]. In order to obtain such fine control over the dynamics within the system, microfluidics is usually coupled with a driving force. Methods described herein utilize electrokinetics (EK) as the driving force as it enables the exploitation of unique behaviors that would not be easily achievable at a larger scale [22], [23].

Accordingly, provided herein are high-throughput microfluidic electrophoresis-based methodologies that exploit the differences in labeling kinetics between dsRNA and mRNA to detect and characterize dsRNA contaminants in mRNA vaccines.

### I. Assay Components

Methods described herein involve separating and detecting dsRNA and ssRNA via capillary electrophoresis in the presence of a polymer solution and an intercalating agent. In some examples, methods described herein can be performed in a microfluidic device.

### Polymer Solutions

The capillary electrophoresis methods described herein involve use of a polymer to separate dsRNA and ssRNA. The polymer can be charged or uncharged. In some embodiments, when the polymer is charged, the charge on the polymer is the same as the charge of the interior surface of the capillary channel. For example, when the capillary channel includes negatively charged groups on the interior surface, the polymer will include monomer subunits that are negatively charged. In another example, when the capillary channel includes positively charged groups on the interior surface, the polymer will include monomer subunits that are positively charged.

Polymers of various percent charge can be used in methods described herein. In some embodiments, the polymer includes a percent charge of between about 0.01% and about 2%, e.g., between about 0.01% and about 1%, between about 0.01% and about 0.5%, between about 0.05% and about 0.5%, and between about 0.05% and about 0.2%.

As used herein, the “percent charge” of a polymer refers to the molar percent of charged monomer units to total monomer subunits used in the synthesis of the polymer. For example, if the polymer synthesis reaction is carried out by mixing 1 mmol of charged subunit and 99 mmol of uncharged monomer subunit, then the polymer would have a percent charge of 1%.

Polymers for use in methods described herein can be formed from various monomer subunits, e.g., negatively charged monomer subunits, positively charged monomer subunits, non-charged monomer subunits, or a combination thereof.

Non-limiting examples of monomer subunits that can be included in a polymer for use in methods described herein include acrylic acid, carboxylic acid, sulfonic acid, phosphoric acid monomer, bisacrylamidoacetic acid, 4,4-Bis(4-hydroxyphenyl)pentanoic acid, 3-butene-1,2,3-tricarboxylic acid, 2-carboxyethylacrylate, itaconic acid, methacrylic acid, and 4-vinylbenzoic acid. In some embodiments, the polymer is an acrylic polymer, e.g., a polyacrylamide polymer, a polymethylacrylamide polymer, a polydimethylacrylamide polymer, and a polydimethylacrylamide-co-acrylic acid polymer.

Non-limiting examples of negatively charged monomer subunits that can be included in a polymer for use in methods described herein include acrylic acid, bisacrylamidoacetic acid, 4,4-Bis(4-hydroxyphenyl)pentanoic acid, 3-butene-1,2,3-tricarboxylic acid, 2-carboxyethylacrylate, itaconic acid, methacrylic acid, 4-vinylbenzoic acid, sulfonic acid, 2-acrylamido-2-methyl-1-propanesulfonic acid, 2-methyl-2-propene-1-sulfonic acid, 2-propene-1-sulfonic acid, 4-styrenesulfonic acid, 2-sulfoethyl methacrylate, 3-sulfopropyldimethyl-3-methacrylamidopropylammonium inner salt, 3-sulfopropyl methacrylate, vinylsulfonic acid, Bis(2-methacryloxyethyl)phosphate, and monoacryloxyethyl phosphate.

Non-limiting examples of positively charged monomer subunits that can be included in a polymer for use in methods described herein include 2-acryloxyethyltrimethylammonium chloride, diallyldimethylammonium chloride, 2-methacryloxyethyltrimethylammonium chloride, and 3-methacryloxy-2-hydroxypropyltrimethylammonium chloride.

Non-limiting examples of polymers that can be used as described herein are provided in U.S. Pat. Nos. 5,948,227; 6,042,710; 6,440,284; and 7,081,190 the relevant disclosures of each of the prior applications are herein incorporated by reference for the purposes and subject matter referenced herein.

The concentration of the polymer in a polymer solution can vary depending on certain factors such as the size of the capillary channel, the size and the amount of the RNA to be analyzed, and/or the desired resolution. In some embodiments, the concentration of polymer in a polymer solution is between about 0.01% to about 30% (w/v), e.g., between about 0.01% to about 20% (w/v), between about 0.01% to about 10% (w/v), between about 0.01% to about 5% (w/v), between about 0.1% to about 30% (w/v), between about 1% to about 30% (w/v), between about 10% to about 30% (w/v), or between about 20% to about 30% (w/v).

The average molecular weight of the polymer in a polymer solution can vary depending on certain factors such as the size of the capillary channel, the size and the amount of the RNA to be analyzed, and/or the desired resolution. In some embodiments, the polymer has a molecular weight between 1 Kd to 5,000 Kd, e.g., between 1 Kd to 2,500 Kd, between 1 Kd to 1,000 Kd, between 1 Kd to 500 Kd, between 1 Kd to 100 Kd, between 100 Kd to 5,000 Kd, between 1,000 Kd to 5,000 Kd, or between 2,500 Kd to 5,000 Kd.

The viscosity of the polymer solution can vary depending on certain factors such as the size of the capillary channel, the size and the amount of the RNA to be analyzed, and/or the desired resolution. In some embodiments, the viscosity of the polymer solution is between about 2 to about 1,000 centipoise, e.g., between about 2 to about 750 centipoise, between about 2 to about 500 centipoise, between about 2 to about 250 centipoise, between about 2 to about 100 centipoise, between about 2 to about 50 centipoise, between about 50 to about 1,000 centipoise, between about 100 to about 1,000 centipoise, between about 250 to about 1,000 centipoise, between about 500 to about 1,000 centipoise, or between about 750 to about 1,000 centipoise.

Synthesis of polymers used in the methods of the present invention may be carried out by any number of methods that are well known in the art. In general, synthesis conditions and protocols will vary depending upon the polymer to be synthesized and the nature and amount of charge to be incorporated. Examples of suitable polymer synthesis methods are described in, e.g., Odian, Principles of Polymerization, Fourth Ed. (John Wiley, New York, 2004), and U.S. Pat. Nos. 5,264,101 and 5,567,292, the relevant disclosures of each of the prior applications are herein incorporated by reference for the purposes and subject matter referenced herein.

### Intercalating Agents

An “intercalating agent” refers to a molecule that can reversibly bind to base pairs in nucleic acids such as DNA (e.g., dsDNA, ssDNA) and/or RNA (e.g., dsRNA, ssRNA). In general, intercalating agents useful in the present methods insert between stacked bases at the center of double-stranded nucleic acids (e.g., dsDNA, dsRNA) or they bind to adjacent bases along the backbone of single-stranded nucleic acids (e.g., ssRNA). As a result, more molecules of the intercalating agent are bound to double-stranded nucleic acids than single-stranded nucleic acids (e.g., dsRNA than ssRNA) at similar concentrations of double-stranded nucleic acids than single-stranded nucleic acids (e.g., dsRNA and ssRNA), which results in a stronger fluorescent signal being produced from double-stranded nucleic acids than single-stranded nucleic acids (e.g., dsRNA than from ssRNA) at similar concentrations. Methods described herein utilize this difference in signal intensity to detect small amounts of dsRNA contaminants in ssRNA samples. Because intercalating agents fluoresce only when bound to dsRNA or ssRNA, the fluorescent signal is specific and indicates the amount of dsRNA or ssRNA in the sample.

The intercalating agent for use in the methods described herein can be any intercalating agent capable of binding to double-stranded nucleic acids and single-stranded nucleic acids (e.g., dsDNA, ssDNA, dsRNA and ssRNA). Non-limiting examples of intercalating agents for use in methods described herein include SYTOTM dyes (e.g., SYTO™ 61), Ribo dyes (e.g., RiboRed, RiboGreen), SYBR dyes, ethidium bromide, acridine orange, propidium iodide, 7-aminoactinomycin D (7-AAD), 4′,6-diamidino-2-phenylindole (DAPI), cyanine dyes (e.g., Cy3, Cy5), Alexa Fluor dyes, BODIPY dyes, and combinations thereof. In some embodiments, the intercalating agent is not ethidium bromide.

Various concentrations of intercalating agent can be used in methods described herein to achieve the desired result. In some embodiments, the concentration of intercalating agent in the sample is between 0.01 mM and 0.2 mM, e.g., between 0.05 mM and 0.2 mM, between 0.1 mM and 0.2 mM, between 0.01 mM and 0.1 mM, or between 0.01 mM and 0.05 mM.

Methods described herein encompass use of an intercalating agent that is intrinsically fluorescent (e.g., an intercalating fluorescent dye) or extrinsically fluorescent (e.g., an intercalating agent labeled with a fluorophore).

In some embodiments, the intercalating agent for use in methods described herein preferentially binds a target nucleic acid. An intercalating agent is said to exhibit “preferential binding” if it reacts or associates more frequently, more rapidly, with greater duration and/or with greater affinity with a particular target nucleic acid than it does with an alternative nucleic acid.

For example, an intercalating agent that preferentially binds to double-stranded nucleic acid is an intercalating agent that binds double-stranded nucleic acid with greater affinity, avidity, more readily, and/or with greater duration than it binds to single-stranded nucleic acid. In another example, an intercalating agent that preferentially binds to single-stranded nucleic acid is an intercalating agent that binds single-stranded nucleic acid with greater affinity, avidity, more readily, and/or with greater duration than it binds to double-stranded nucleic acid.

For example, an intercalating agent that preferentially binds to DNA is an intercalating agent that binds DNA with greater affinity, avidity, more readily, and/or with greater duration than it binds to RNA. In another example, an intercalating agent that preferentially binds to RNA is an intercalating agent that binds RNA with greater affinity, avidity, more readily, and/or with greater duration than it binds to DNA.

It is also understood that an intercalating agent that preferentially binds to a first nucleic acid (e.g., DNA) may or may not preferentially bind to a second nucleic acid (e.g., RNA). As such, preferential binding does not necessarily require (although it can include) exclusive binding.

Methods described herein encompass use of one or more intercalating agents. For example, methods described herein can include use of an intercalating agent capable of binding to RNA and an intercalating agent capable of binding to DNA.

### II. Methods for Separating and Detecting dsRNA and ssRNA

Also provided herein are methods for separating and detecting small amounts of dsRNA that often contaminate ssRNA preparations including RNA therapies (e.g., mRNA therapies such as mRNA vaccines). Methods described herein involve capillary electrophoresis of samples of dsRNA and ssRNA in the presence of a polymer and an intercalating agent, each of which is disclosed herein.

To perform methods described herein, in some embodiments, a polymer solution is introduced into a capillary channel and then a sample comprising dsRNA and ssRNA is introduced into the capillary channel. An intercalating agent for detecting dsRNA and ssRNA can be included in the polymer solution or in the sample. This introduction can be as simple as placing one end of the channel into contact with the polymer solution or the sample and allowing the polymer solution or the sample to wick into the channel. Alternatively or in addition to, vacuum or pressure can be used to drive the polymer solution or the sample into the capillary channel.

Capillary electrophoresis methods described herein can be performed using a single capillary channel or multiple capillary channels. In some embodiments, when using a single channel, the sample is introduced into the capillary channel (e.g., by hydrodynamic injection) and voltage is applied across the ends of the capillary channel to separate dsRNA and ssRNA in the sample.

In some embodiments, when using multiple channels, the sample is introduced into a first capillary channel (also referred to as a loading channel) and transported across an intersection of the first channel with a second capillary channel (also referred to as an analysis channel). The volume or ‘plug’ of sample that is disposed within the intersection of the two channels is then drawn down the second channel where it is subjected to the desired analysis.

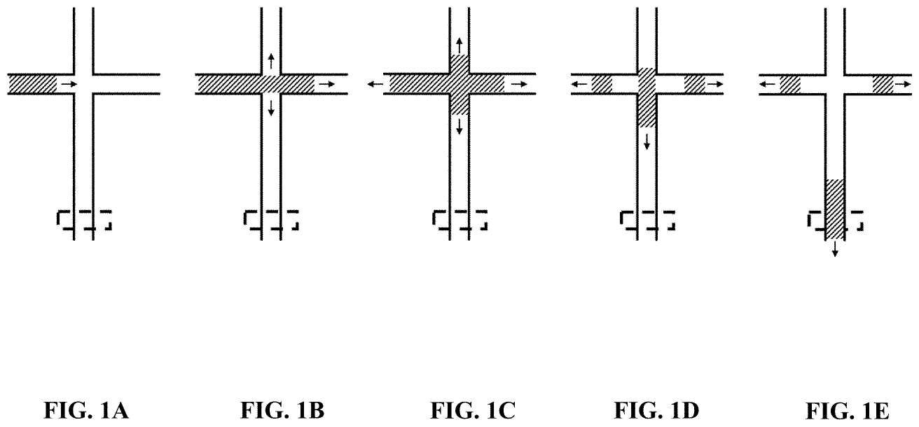

FIGS. 1A-1E schematically illustrate loading of the sample (indicated by hatching ///) into the loading channel and injection of the sample into the analysis channel. As shown in FIGS. 1A-1B, the sample is drawn into the loading channel and across the intersection with the analysis channel. As shown in FIGS. 1B-1C, transport of sample across the intersection without diffusing into the analysis channel is accomplished by applying a slight voltage gradient away from the flow path and toward the top and bottom termini of the analysis channel. The result is a ‘pinching’ of the sample flow at the intersection, which prevents the diffusion of the sample into the analysis channel. As shown in FIG. 1D, the pinched volume of sample at the intersection can then be injected into the analysis channel by applying a voltage gradient across the length of the analysis channel, e.g., from the top terminus to the bottom terminus. In order to avoid any bleeding over of material from the sample loading channel during this injection, a low level of flow is directed back into the side channels, resulting in a ‘pull back’ of the material from the intersection as shown FIGS. 1D-1E. The sample is electrophoresed through the analysis channel and flowed past the detection window (dashed box) as shown in FIG. 1E. When using a fluorescent intercalating agent, a laser activated fluorescent detection system can be used to monitor the detection window for a fluorescent signal produced by the intercalating agent bound to dsRNA or ssRNA.

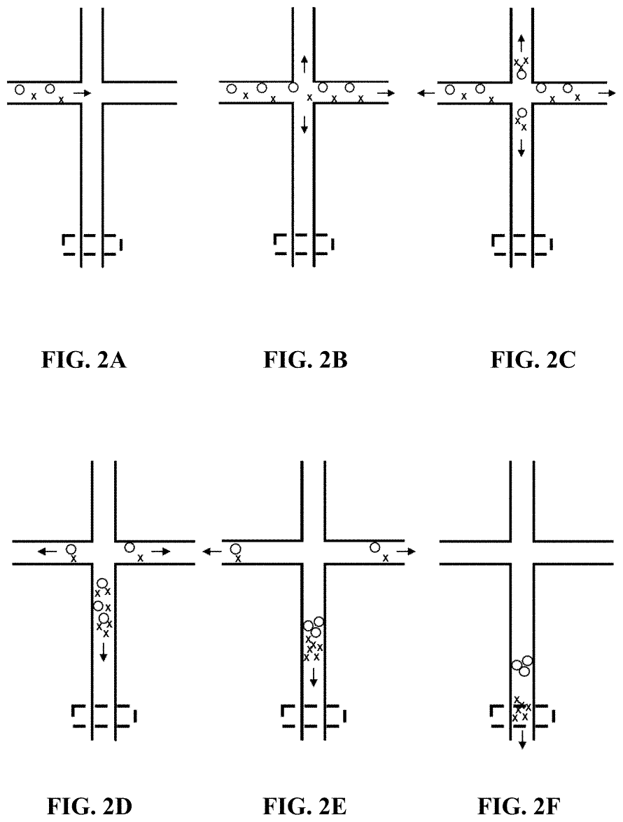

When the sample is loaded and injected as schematically illustrated in FIGS. 1A-1E, dsRNA can be separated from ssRNA and then the separated dsRNA and ssRNA can be sequentially detected as schematically illustrated in FIGS. 2A-2F.

As shown in FIGS. 2A-2B, the sample is drawn into the loading channel and across the intersection with the analysis channel, thereby aligning dsRNA (indicated by Xs) and ssRNA (indicated by Os) in the applied electric field. FIGS. 2B-2C show ‘pinching’ of the sample at the intersection by applying a slight voltage gradient away from the flow path and toward the top and bottom termini of the analysis channel. As shown in FIG. 2D, the pinched volume of sample at the intersection, which includes both dsRNA and ssRNA, is then injected into the analysis channel by applying a voltage gradient across the length of the analysis channel, e.g., from the top terminus to the bottom terminus. As shown in FIGS. 2E-2F, the sample is electrophoresed through the analysis channel such that faster migrating dsRNA is separated from slower migrating ssRNA, which allows dsRNA to flow past the detection window (dashed box) separately and prior to the flow of ssRNA past the detection window.

In some embodiments, the polymer solution can be introduced or ‘preloaded’ into the capillary channel prior to introducing the sample of dsRNA and ssRNA into the capillary channel. As such, the sample is introduced into a capillary channel preloaded with the polymer solution. In such instances, the polymer solution and/or the sample can include the intercalating agent.

The sample can be in an aqueous buffer having a similar or a different ionic strength as the polymer solution. In some embodiments, the sample is in an aqueous buffer having a similar ionic strength as that of the polymer solution. In some embodiments, the sample is in an aqueous buffer having a lower ionic strength compared to the ionic strength of the polymer solution.

Methods described herein encompass detecting dsRNA and ssRNA, or a lack thereof, in various samples. In some embodiments, the sample is a RNA-based therapeutic. In such instances, methods provided herein can be used for evaluating purity of a RNA-based therapeutic, e.g., detect amount of dsRNA contaminants in a mRNA therapy. Non-limiting examples of RNA-based therapeutics that can be analyzed as described herein include antisense oligonucleotides, aptamers, small interfering RNAs, microRNAs, and messenger RNAs.

Methods described herein can be used to detect low concentrations of dsRNA and ssRNA in a sample. In some examples, the limit of detection (LOD) of methods described herein can be between 1 pg/μL and 100 pg/μL for dsRNA and ssRNA.

Accordingly, in some examples, the concentration of dsRNAs in the sample is between 10 pg/μL and 200 ng/μL, e.g., between 50 pg/μL and 200 ng/μL, between 100 pg/μL and 200 ng/μL, between 150 pg/μL and 200 ng/μL, between 10 pg/μL and 150 ng/μL, between 10 pg/μL and 100 ng/μL, or between 10 pg/μL and 50 ng/μL. In some examples, the concentration of dsRNAs in the sample is between 1 pg/μL and 100 pg/μL , e.g., between 10 pg/μL and 100 pg/μL , between 25 pg/μL and 100 pg/μL, between 50 pg/μL and 100 pg/μL, between 75 pg/μL and 100 pg/μL, between 1 pg/μL and 75 pg/μL, between 1 pg/μL and 50 pg/μL, between 1 pg/μL and 25 pg/μL, or between 1 pg/μL and 10 pg/μL.

In some examples, the concentration of ssRNAs in the sample is between 50 pg/μL and 200 ng/μL, e.g., between 100 pg/μL and 200 ng/μL, between 150 pg/μL and 200 ng/μL, between 50 pg/μL and 150 ng/μL, or between 50 pg/μL and 100 ng/μL. In some examples, the concentration of ssRNAs in the sample is between 1 pg/μL and 100 pg/μL, e.g., between 10 pg/μL and 100 pg/μL, between 25 pg/μL and 100 pg/μL, between 50 pg/μL and 100 pg/μL, between 75 pg/μL and 100 pg/μL, between 1 pg/μL and 75 pg/μL, between 1 pg/μL and 50 pg/μL, between 1 pg/μL and 25 pg/μL, or between 1 pg/μL and 10 pg/μL.

In some examples, the percent of dsRNA contaminants relative to the total amount of ssRNA in the sample is between 0.1% and 1%, e.g., between 0.25% and 1%, between 0.5% and 1%, between 0.75% and 1%, between 0.1% and 0.75%, between 0.1% and 0.5%, or between 0.1% and 0.25%.

Methods described herein can be carried out using various microfluidic devices and systems. A microfluidic device or microfluidic system refers to a device or system which incorporates at least two intersecting channels or fluid conduits, where at least one of the channels has at least one cross sectional dimension in the range of from about 0.1 to about 500 μm, e.g., about 1 to about 100 μm.

Microfluidic devices for use in methods described herein can include a central body structure in which the various microfluidic elements are disposed. For example, the body structures of the microfluidic devices for use in methods described herein typically employ a solid or semi-solid substrate that is typically planar in structure, e.g., substantially flat or having at least one flat surface. Suitable substrates may be fabricated from any one of a variety of materials, or combinations of materials. Often, substrates are manufactured using solid substrates common in the fields of microfabrication, e.g., silica, silicon or gallium arsenide. In the case of these substrates, common microfabrication techniques, such as photolithographic techniques, wet chemical etching, micromachining can be readily applied in the fabrication of microfluidic devices and substrates. Alternatively, polymeric substrate materials may be used to fabricate the devices for use in methods described herein, including, e.g., polydimethylsiloxanes (PDMS), polymethylmethacrylate (PMMA), polyurethane, polyvinylchloride (PVC), polystyrene, polysulfone, polycarbonate and the like.

In general, microfluidic devices for use in methods described herein include an analysis channel and a loading channel in a cross-channel structure, a sample is loaded into the analysis channel by placing it in a reservoir at the terminus of the loading channel and applying a voltage across the loading channel until the sample has electrophoresed across the intersection of loading channel and the analysis channel. Typically, the application of voltage is via an electrode disposed within the reservoir or well at the terminus of the given channel (also referred to as a “port”). The plug of sample at the intersection is then electrophoresed down the analysis channel by applying a voltage across the analysis channel.

Examples of suitable microfluidic systems, methods, and devices are described in, e.g., U.S. Pat. Nos. 5,976,336; 7,419,784; 7,276,330; 7,081,190; 5,948,227; 6,042,710; 6,440,284, each of which is incorporated herein by reference in its entirety.

### EXAMPLES

In order that the invention described may be more fully understood, the following examples are set forth. The examples described in this application are offered to illustrate the methods and compositions provided herein and are not to be construed in any way as limiting their scope.

### Example 1: Separation and Detection of dsRNA in Samples Including dsRNA and ssRNA

Separation and detection of standard dsRNA and ssRNA samples in a microscale electrophoresis device (LabChip® GX/GXII Touch™ instrument) were performed as follows.

A. Preparation of the Reagent Chip

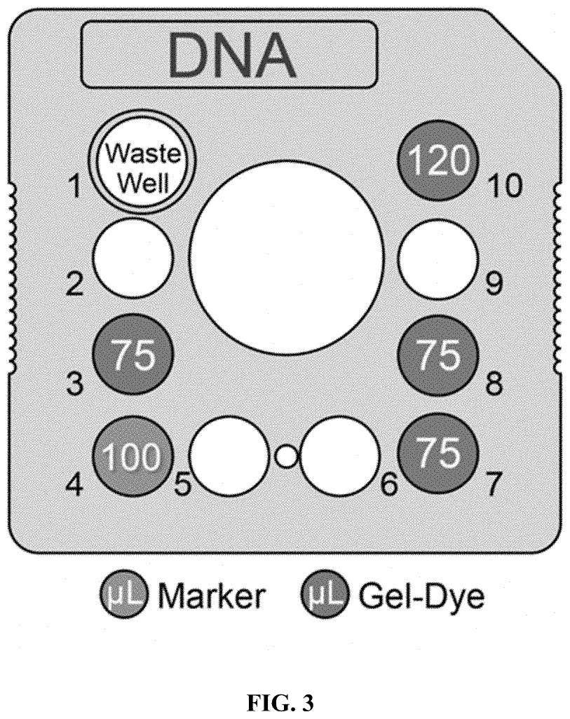

A polymer-dye mixture was prepared by adding 5504 of polymer to 7.5 μL of dye. The polymer-dye mixture was filtered using a spin filter and centrifugation at 9200 rcf for 7.5 minutes a room temperature. Then, the reagent chip was loaded by adding 75 μL of the polymer-dye mixture to the wells 3, 7, and 8, and 120 μL of the polymer-dye mixture to well 10. The reagent chip was also loaded with 100 μL of marker solution (DNA High Sensitivity Reagent Kit, PN CLS760672) in well 4. See FIG. 3 for placement of the reagents on the chip.

B. Separation of dsRNA ladder

Separations were performed on a dsRNA ladder (New England Biolab, Cat# N0363S) which includes a set of 7 annealed double-stranded RNA molecules produced by in vitro transcription of 14 linear DNA templates. The ladder sizes are: 500, 300, 150, 80, 50, 30 and 21 base pairs.

The dsRNA sample was prepared by adding 64 of dsRNA ladder to 114 μA of DI water to form a 1× ladder solution. Then, 24 of the 1× ladder and 8μL of DI water were added to the well of the sample plate. Three wells were loaded with sample and the analysis was performed in triplicate.

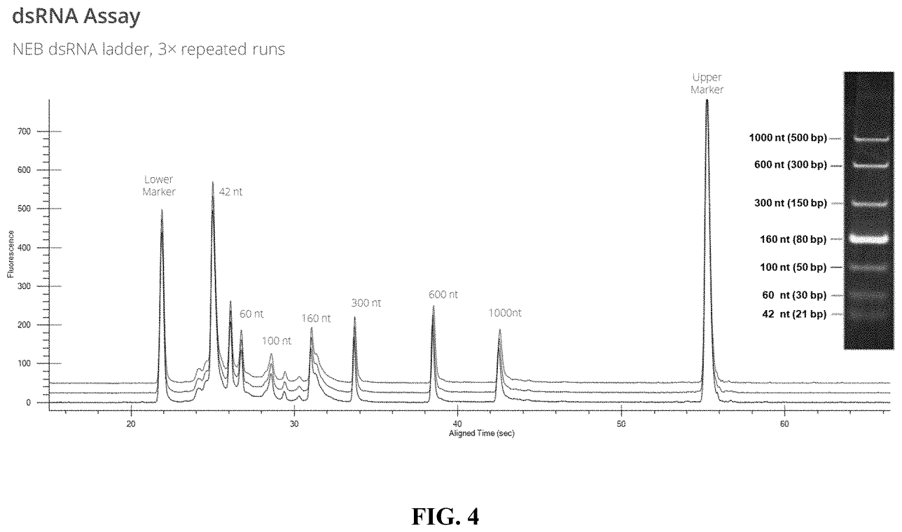

Sample and polymer-dye mixture were mixed and run through the separation channel of the microfluidic device. Fluorescence was detected at the detection window. A representative graph of fluorescence versus time for each sample is shown in FIG. 4. As shown, each dsRNA in the ladder (500, 300, 150, 80, 50, 30 and 21 base pairs) was separated and detected.

C. Separation of ssRNA ladder

Separations were performed on a ssRNA ladder (New England Biolab, Cat# N0364S) which includes a set of 6 RNA molecules produced by in vitro transcription of a mixture of 6 linear DNA templates. The ladder sizes are: 1000, 500, 300, 150, 80 and 50 bases.

The ssRNA sample was prepared as described for the dsRNA ladder. Specifically, 6 μL of ssRNA ladder was added to 114 μL of DI water to form a 1× ladder solution. Then, 2 μL of the 1× ladder and 8μL of DI water were added to the well of the sample plate. Three wells were loaded with sample and the analysis was performed in triplicate.

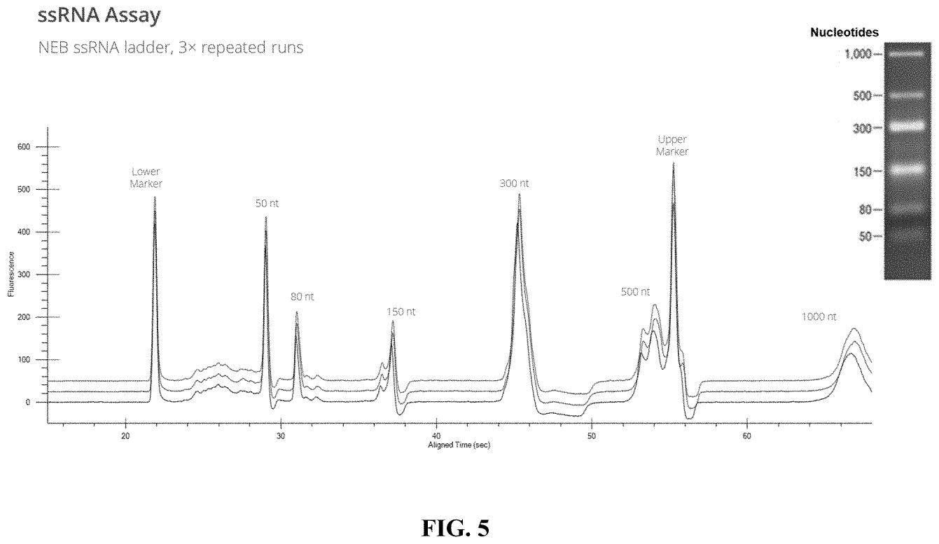

Sample and polymer-dye mixture were mixed and run through the separation channel of the microfluidic device. Fluorescence was detected at the detection window. A representative graph of fluorescence versus time for each sample is shown in FIG. 5. As shown, each dsRNA in the ladder (1000, 500, 300, 150, 80 and 50 bases) was separated and detected.

D. Separation of dsRNA and ssRNA Mixtures

Separations will be performed on mixtures of the dsRNA ladder (New England

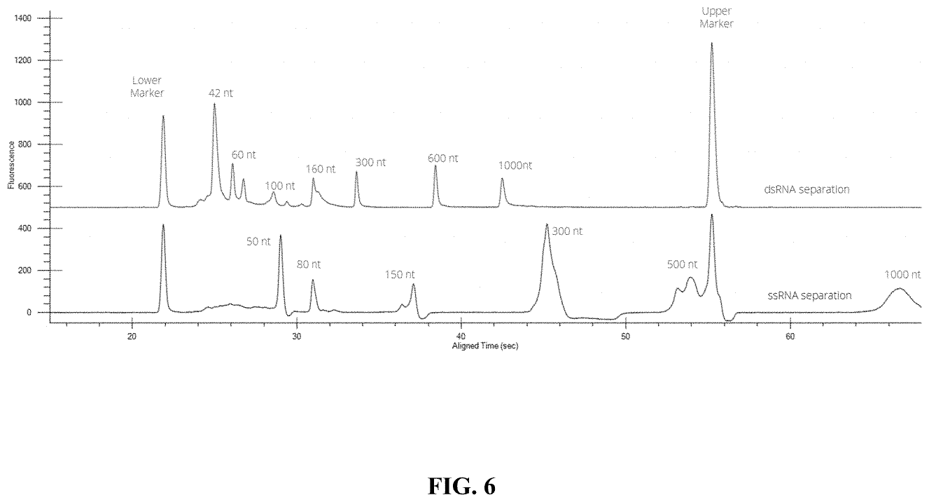

Biolab, Cat# N0363S) and ssRNA ladder (New England Biolab, Cat# N0364S). Sample and polymer-dye mixture will be mixed and run through the separation channel of the microfluidic device. Fluorescence will be detected at the detection window. A representative graph of fluorescence versus time for the dsRNA ladder and the ssRNA ladder are shown in FIG. 6. As shown, at the same molecular weight, dsRNA migrates faster than ssRNA, e.g., 300 nt (150 bp) dsRNA migrates faster than 300 nt ssRNA (˜34 seconds compared to ˜45 seconds). As also shown, at similar concentrations, ssRNA (7.14 ng/μL) exhibited lower fluorescence signal than dsRNA (6.25 ng/4) (0-400 fluorescence units compared to 500-1000 fluorescence units).

### Example 2: Increased Sensitivity of dsRNA Detection from Improved Pinch Flow

The low abundance of dsRNA contaminants in ssRNA samples makes detection of dsRNA difficult. Therefore, an improved pinch flow technique was developed to improve the sensitivity of dsRNA detection. Separation and detection of a standard dsRNA sample in a microscale electrophoresis device (LabChip® GX/GXII Touch™ instrument) was performed using standard pinch flow conditions and improved conditions. The standard and improved pinch flow conditions used the same voltages to drive the sample in the load and separation channels. The improved pinch flow conditions utilized a longer load time and a higher current to allow injection of a larger plug of sample into the separation channel. As shown in

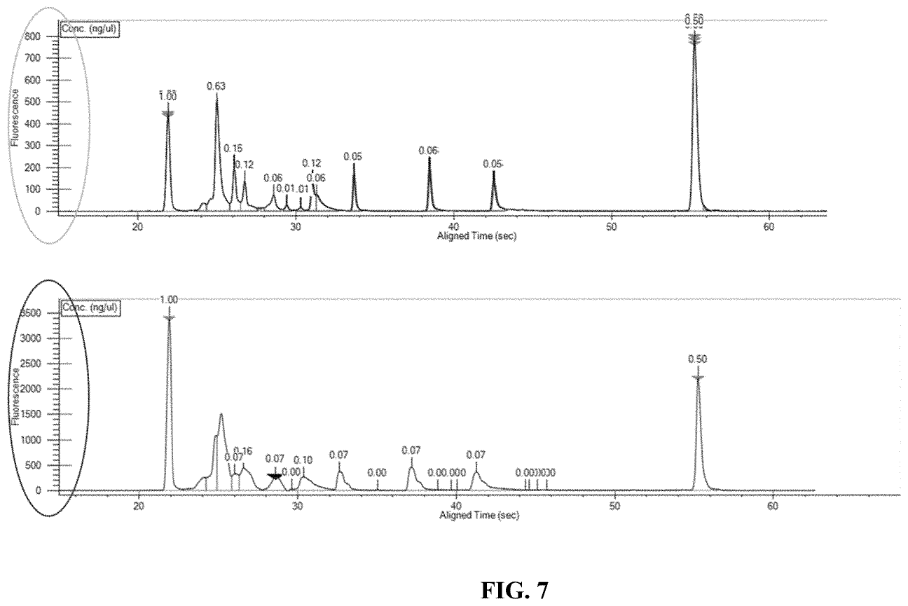

FIG. 7, the fluorescent signal was increased about 6-fold in the chromatographic separation of the dsRNA standard sample using an improved pinch flow (bottom panel) compared to the fluorescent signal using the standard pinch flow (top panel).

### Example 3: Development of an Assay for Detecting dsRNA Contaminants in ssRNA Samples

This Example describes development of an assay for detection and characterization of harmful dsRNA contaminant in mRNA vaccines using a dsRNA ladder to simulate dsRNA contaminants and a ssRNA ladder to simulate mRNA. Ladders were used since long dsRNA and mRNA molecules are not readily available, in particular in a highly purified form, and limited data has been collected to characterize them. The findings from these experiments were then used as a reference or starting point for the analysis of custom ordered long (4,001 bp or nt) dsRNA and mRNA samples.

The ssRNA (catalogue #N0364S) and dsRNA ladders (catalogue #N0363S) used in experiments described herein were purchased from New England Biolabs (New England Biolabs, Ipswich, Mass.). The ssRNA ladder contains fragments of 50 nt, 80 nt, 150 nt, 300 nt, 500 nt and 1,000 nt, while the dsRNA ladder contains fragments of 21 bp, 30 bp, 50 bp, 80 bp, 150 bp, 300 bp and 500 bp. We note that the fragment concentration in the ladders, particularly the dsRNA ladder, has not been fully characterized by the provider since difficulties in synthesis can lead to significant batch-to-batch differences.

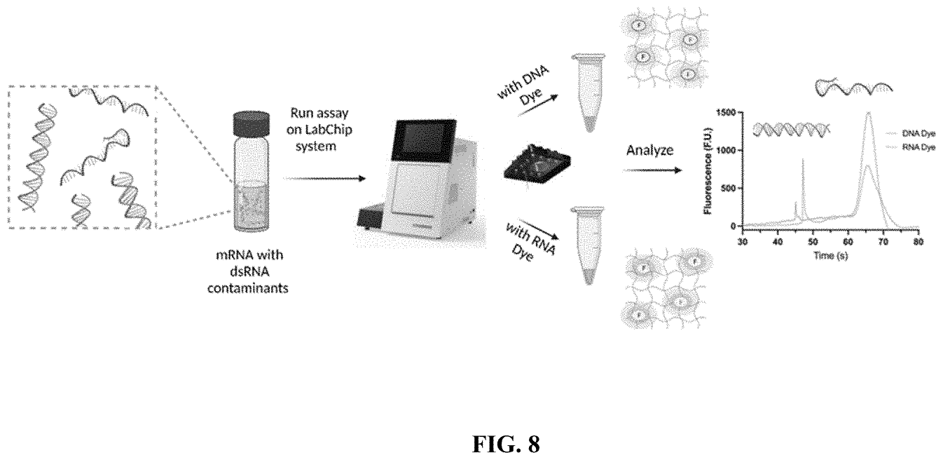

Assays were performed using the high throughput LabChip GX Touch II platform (PerkinElmer, Waltham, Mass.). As seen in FIG. 8, samples were analyzed in a chip loaded with a DNA dye gel matrix that includes polydimethysiloxane (PDMS) and SYTO™ 61, and upon completion, the same sample were analyzed using the same chip loaded with an RNA dye gel matrix that includes PDMS and RiboRed. Once both assays have been run, the results were analyzed as described herein. Assays were performed using different fluorescent dyes (SYTO™ 61 and RiboRed; PerkinElmer) as well as varying concentrations of the fluorescent dyes and gel polymers. Similar results were obtained regardless of the order of sample analysis with the DNA dye gel matrix and the RNA dye gel matrix.

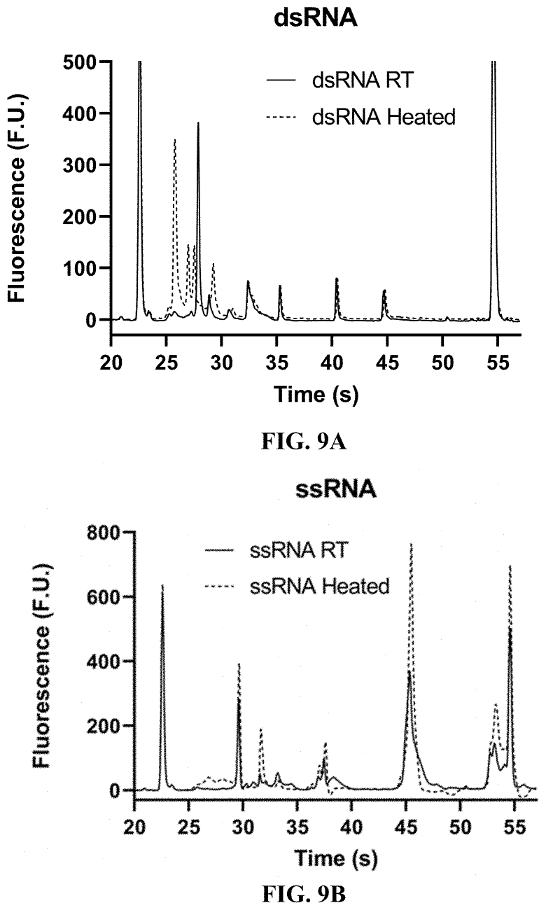

Single-stranded RNA molecules are generally heated prior to analysis to prevent their tertiary structure (e.g., hairpins) from interfering with the accurate measurement of their mobility. However, it was observed that the use of heat denatured smaller strands of dsRNA, which caused the fabrication of dsRNA peaks (FIG. 9A). Heating resulted in defined peaks for the ssRNA sample (FIG. 9B). Thus, assays described herein exclude heating from the sample preparation because the denaturing caused by heating the sample made dsRNA more difficult to characterize.

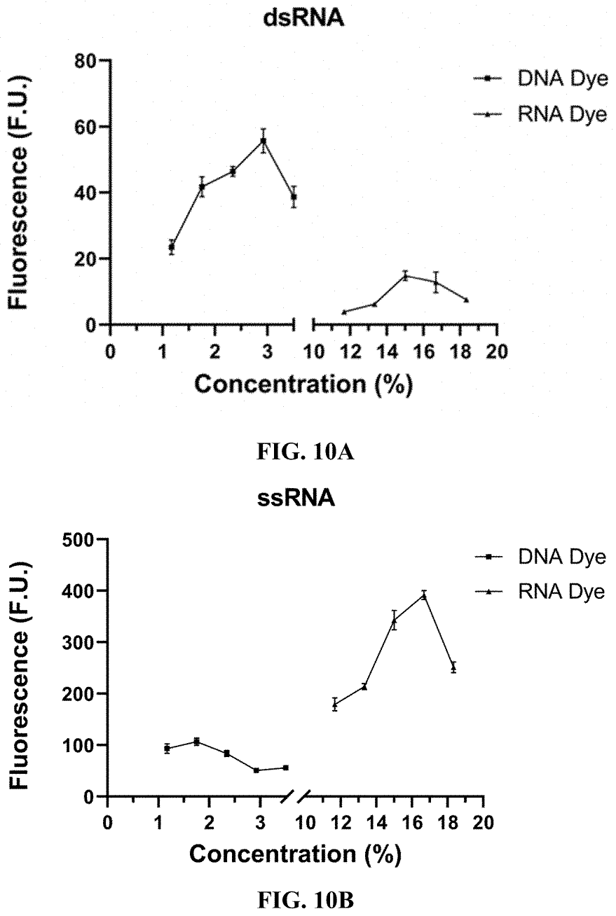

Dye concentrations were varied and DMSO was used to keep the gel matrix to dye volume ratio constant for a given dye type. The DNA dye (SYTO™ 61; PerkinElmer) was diluted to a concentration of 0.58%, 1.17%, 1.75%, 2.34%, and 2.95% and the RNA dye (RiboRed; PerkinElmer) was diluted to a concentration of 11.67%, 13.33%, 15.00%, 16.67%, and 18.33%. These percentages are concentrations based on the concentrations provided for stock vials of the dyes, and therefore the percentages are not indicative of the molarity of the solutions. Experiments were performed using 50 bp or nt fragments for each ladder, which served as a representative fragment size, and a 6% gel matrix. At the concentrations tested, increased fluorescence was observed for dsRNA using the DNA dye (FIG. 10A) and for ssRNA using the RNA dye (FIG. 10B).

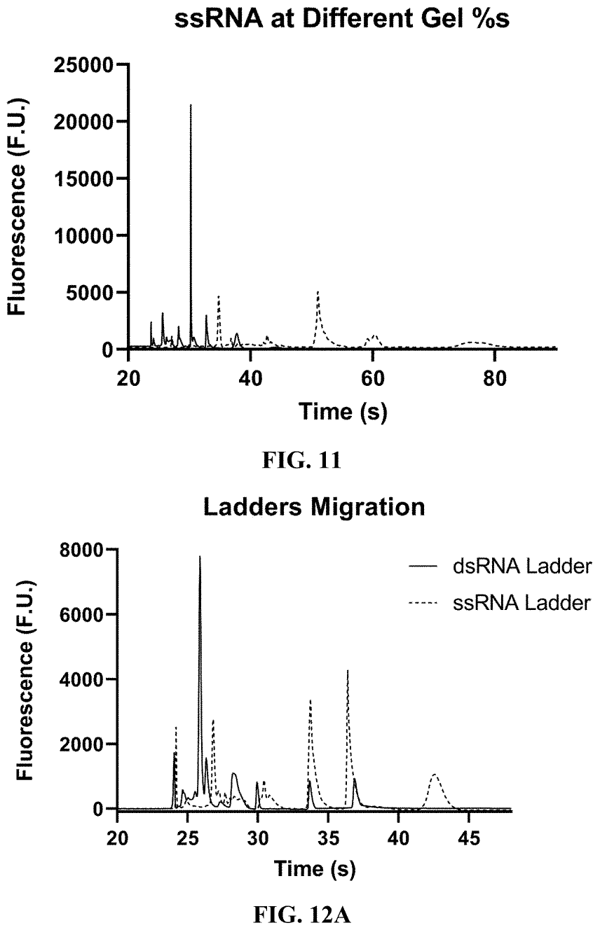

The ssRNA ladder was analyzed using 6% gel and then using a 3% gel matrix mixture. The ladder showed a significantly greater spread of the peaks at 6% gel than at 3% gel (FIG. 11), which may be due in part to the significantly slower migration using the 6% gel. The dsRNA ladder was also assessed with both gel percentages and similar results were obtained, however, the absence of a larger fragment such as the 1,000 nt present in the ssRNA ladder made the changes in separation less apparent for the dsRNA ladder (data not shown).

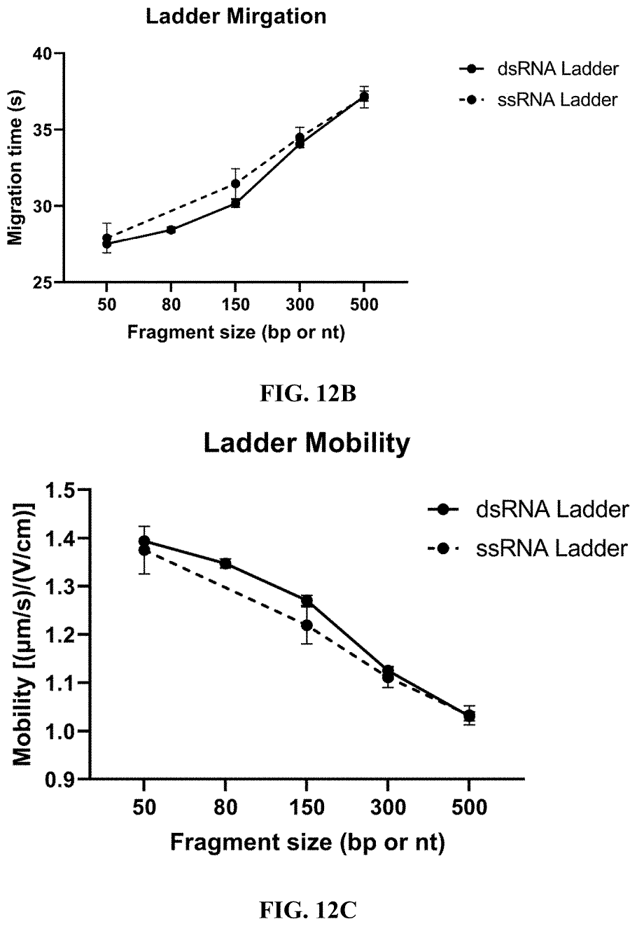

The dsRNA and ssRNA ladders were analyzed using the microfluidic electrophoresis chip platform and electropherograms were obtained (FIG. 12A). To determine whether there were any differences between the two electropherograms, we assessed whether there was a significant difference between the migration time (FIG. 12B) and mobility (FIG. 12C) between the two types of molecules (Table 1). However, no significant differences in migration time and mobility were observed (FIGS. 12B-12C).

TABLE 1
Comparison of the migration of different fragments in a dsRNA and a
ssRNA ladder within a microfluidic platform. Experiments were
conducted using a DNA dye and 3% gel matrix.

| Fragment | dsRNA | dsRNA | SsRNA | mRNA |
|---|---|---|---|---|
| size | migration | mobility | migration | mobility |
| (bp or nt) | time (s) | ([μm/s]/[V/cm]) | time (s) | ([μm/s]/[V/cm]) |
| 50 | 24.5 ± 0.2 | 1.39 ± 0.01 | 27.9 ± 1.0 | 1.38 ± 0.05 |
| 80 | 28.5 ± 0.2 | 1.35 ± 0.01 | * | * |
| 150 | 30.2 ± 0.3 | 1.27 ± 0.01 | 31.47 ± 1.0 | 1.22 ± 0.04 |
| 300 | 34.1 ± 0.3 | 1.13 ± 0.01 | 34.5 ± 0.7 | 1.11 ± 0.02 |
| 500 | 37.2 ± 0.3 | 1.03 ± 0.01 | 37.1 ± 0.7 | 1.03 ± 0.02 |

* Note
that some fragments were excluded from the analysis due to difficulty distinguishing it from other fragments or from the noise.

### Example 4: Using DNA and RNA Dyes to Label dsRNA and ssRNA Fragments

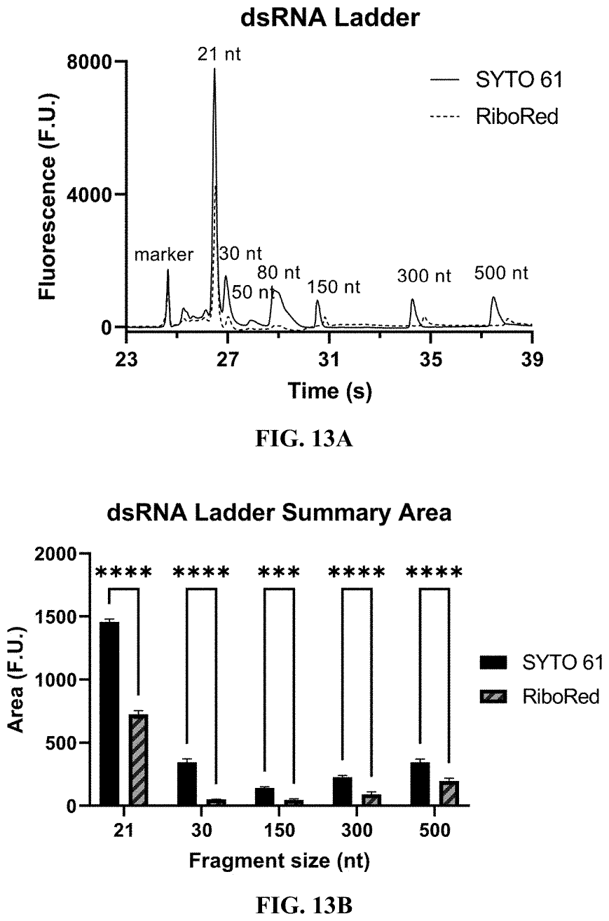

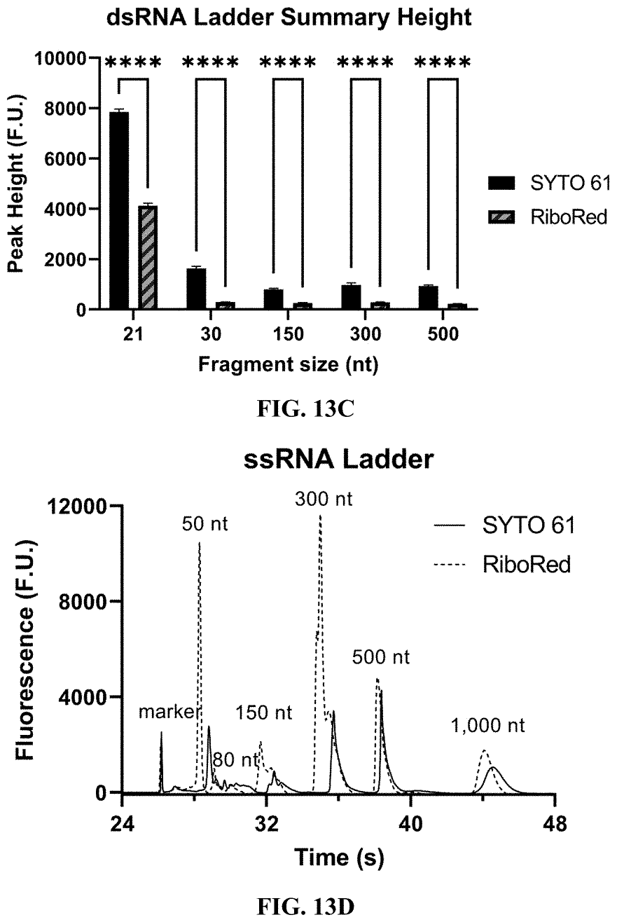

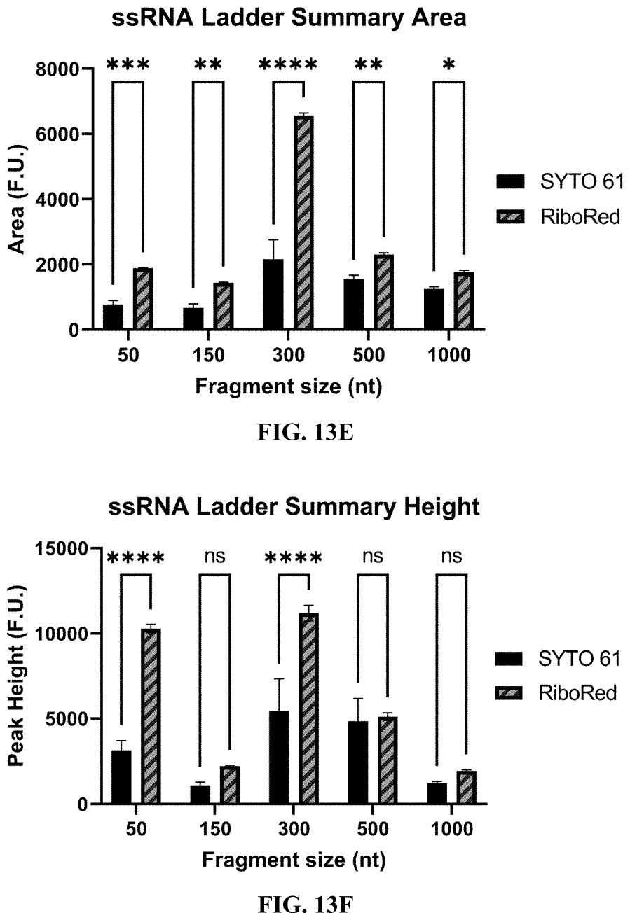

Due to the overlaps in structure between dsRNA and dsDNA and in bases between dsRNA and ssRNA, we explored both a DNA dye and an RNA dye for the visualization of our molecules. The DNA dye and the RNA dye used in the experiments were SYTO™ 61 and RiboRed, respectively. To do so, a new experiment was conducted where a dsRNA and an ssRNA ladder were analyzed in a chip loaded with a gel matrix that contained DNA dye and then the same samples were analyzed in the same chip loaded with a gel matrix that contained RNA dye. Similar to the previous results, we noticed that the overall fluorescence of the ssRNA peaks were higher than those of the dsRNA peaks, but more interestingly than that, we noticed that the way the ladders responded to the dyes was opposite to each other. While dsRNA ladder fragments generally produced lower degrees of fluorescence than ssRNA ladder fragments when labeled with both dyes, we noticed the following trend: dsRNA exposed to a DNA dye was labeled better than dsRNA exposed to a RNA dye while ssRNA was labeled better with the RNA dye (FIGS. 13A-13F, Tables 2-3). As discussed below, this difference in response may be due to the overall structural difference of both types of nucleic acid molecules and how the respective fluorophores can fit within their conformations.

TABLE 2
Comparison of the dsRNA fragment peak areas and heights using
different dye types. Experiments were conducted using a DNA dye at
1.2%, an RNA dye at 15.0%, when applicable, and a 3% gel matrix.

| Fragment | Area | | Height | |
|---|---|---|---|---|
| size | Area DNA dye | Area RNA | Height DNA | Height RNA |
| (bp or nt) | (F.U.) | dye (F.U.) | dye (F.U.) | dye (F.U.) |
| 21 | 1458.0 ± 22.8 | 724.2 ± 29.7 | 7855.5 ± 114.3 | 4129.8 ± 102.9 |
| 30 | 345.0 ± 27.5 | 51.3 ± 1.7 | 1628.6 ± 91.7 | 299.3 ± 4.8 |
| 150 | 142.9 ± 7.8 | 46.7 ± 9.8 | 804.9 ± 37.9 | 255.8 ± 26.0 |
| 300 | 226.5 ± 14.9 | 90.9 ± 19.9 | 967.8 ± 97.8 | 284.4 ± 22.1 |
| 500 | 346.6 ± 25.0 | 196.1 ± 22.8 | 930.7 ± 50.9 | 226.2 ± 14.2 |

TABLE 3
Comparison of the ssRNA fragment peak areas and heights using
different dye types. Experiments were conducted using a DNA dye at
1.2%, an RNA dye at 15.0%, when applicable, and a 3% gel matrix.

| Frag- | Area | | Height | |
|---|---|---|---|---|
| ment | Area DNA | Area RNA | Height DNA | Height RNA |
| size (bp | dye | dye | dye | dye |
| or nt) | (F.U.) | (F.U.) | (F.U.) | (F.U.) |
| 50 | 772.7 ± 124.8 | 1883.5 ± 12.9 | 31341 ± 568.7 | 10287.0 ± 244.9 |
| 150 | 662.2 ± 128.3 | 1434.8 ± 23.4 | 1091.3 ± 191.5 | 2214.7 ± 58.6 |
| 300 | 2157.5 ± 596.9 | 6566.3 ± 80.1 | 5423.5 ± 1918.5 | 11187.7 ± 451.4 |
| 500 | 1565.7 ± 104.7 | 2296.8 ± 59.5 | 4845.2 ± 1319.0 | 5103.6 ± 226.6 |
| 1000 | 1246.9 ± 66.7 | 1761.1 ± 60.9 | 1197.8 ± 122.7 | 1932.8 ± 68.4 |

When comparing the areas yielded using the DNA dye to those using the RNA dye, on average, the dsRNA ladder saw a decrease in area of 56% while the ssRNA ladder saw an increase in area of 118%. Interestingly, while the same trend was observed in both the peak area and peak height for each fragment size, it was noted that peak heights failed to show a statistical difference for three of the five ssRNA fragments analyzed. While the standard deviations were higher in the case of the peak heights, the changes in peak height when comparing those yielded with the DNA dye to those yielded with the RNA dye showed similar values; a 57% decrease for the dsRNA ladder and a 96% increase for the ssRNA ladder. This suggests that while both parameters are in agreement, peak areas, which on average displayed smaller error bars, can be used as an indicator when assessing the effect of different dyes or conditions.

### 5 Example 5: Assay Performance Using Long dsRNA and mRNA Fragments

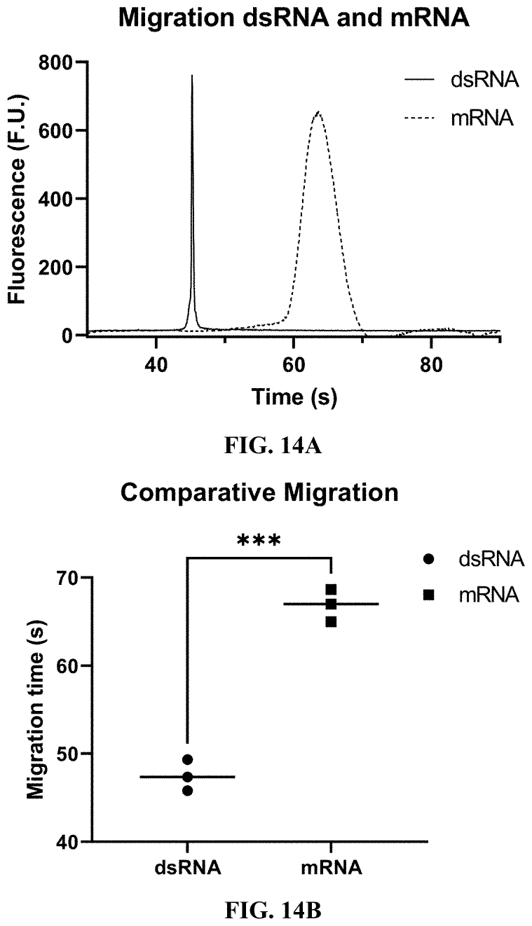

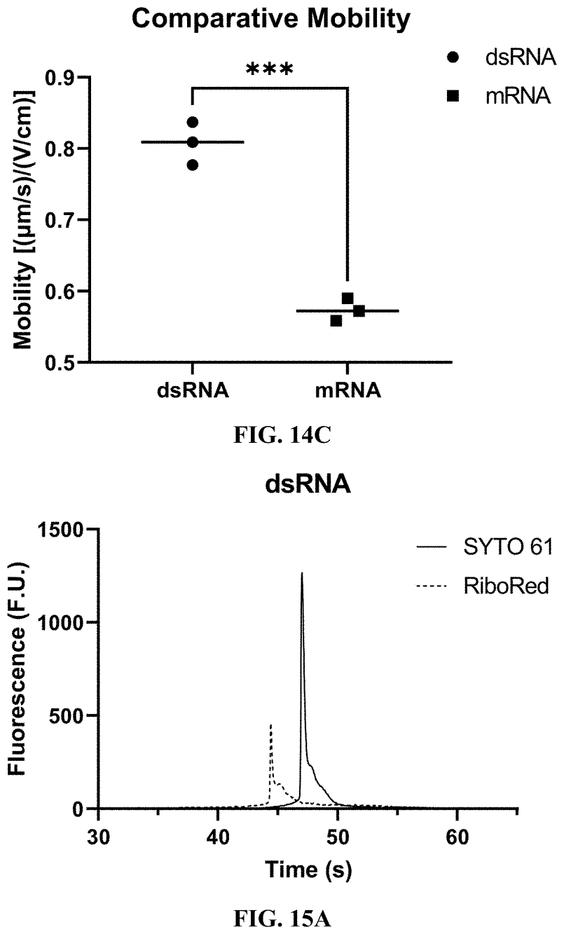

To assess whether the findings made using the shorter dsRNA (<500 bp) and ssRNA fragments (<1,000 nt) were also applicable to long fragments (4,001 bp or nt), we analyzed a long dsRNA and a long mRNA molecule using a DNA dye and an RNA dye, which were SYTO™ 61 and RiboRed, respectively. These molecules were custom-ordered to have the same sequences in order to reduce any effect that could be caused by differences in sequence (e.g., charge, flexibility). Interestingly, while FIG. 12C suggests that there is not a significant difference in the mobility of shorter ssRNA and dsRNA fragments, FIGS. 14A-14C suggests that there is a significant difference in mobility between long dsRNA and mRNA molecules. This difference could be due to the length of the molecule or the use of mRNA instead of ssRNA. It was also shown that, despite the dsRNA and mRNA having similar concentrations, like in experiments performed using shorter fragments, they showed a significant difference in the area under the curve. Here, the dsRNA was analyzed at a concentration of 2.60 ng/μL, while the mRNA was used at a concentration of 5.90/μL; however, instead of showing an increase in area equivalent to the change in concentration between the two, which is 2.3 times, on average the area of the mRNA sample was 9.6 times greater than that of the dsRNA sample. Therefore, we evaluated how each molecule type responded to distinct types of dye to better understand the mechanism behind this disproportionate change.

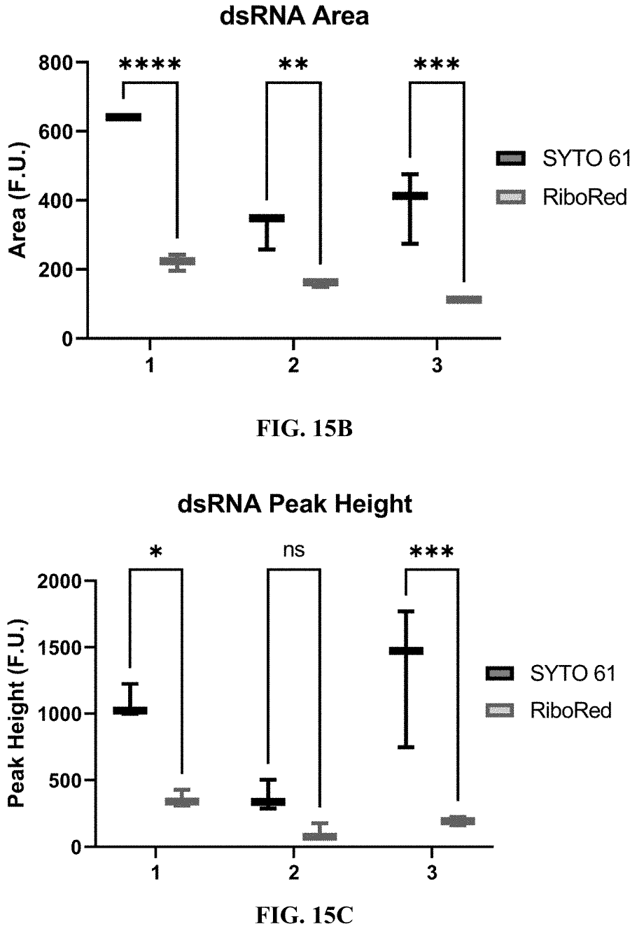

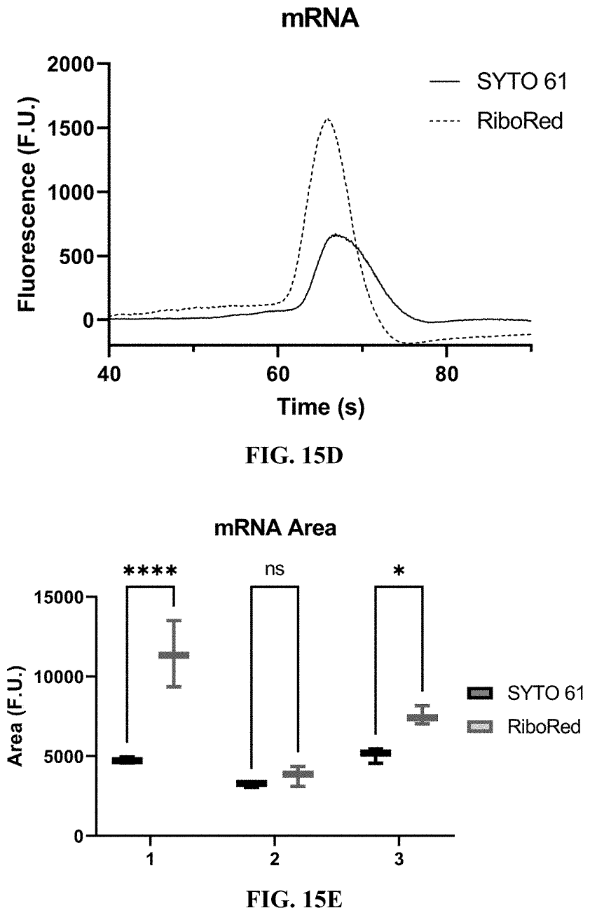

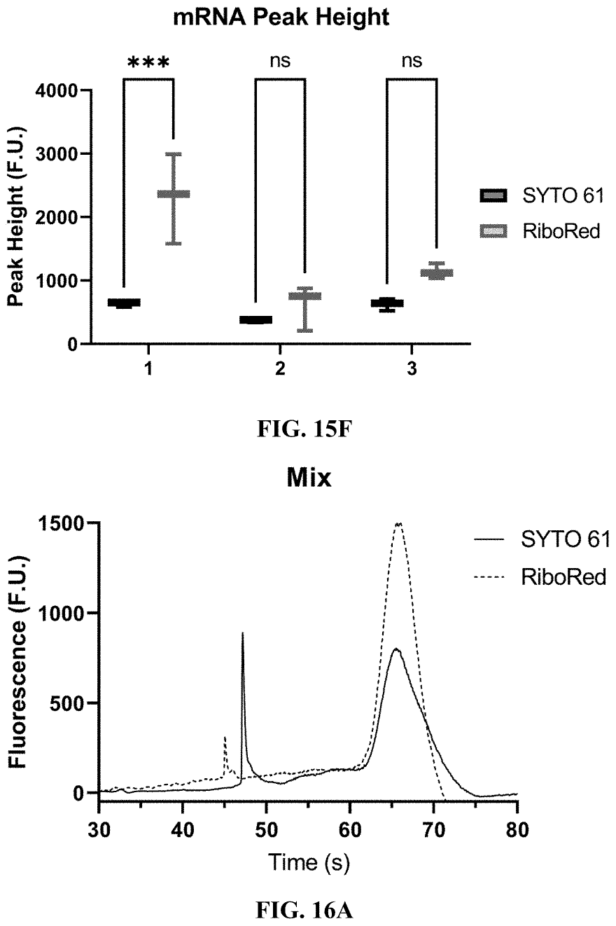

When the custom, long dsRNA and mRNA were analyzed using both dye types (FIGS. 15A-15C, Tables 4-5), we observed a difference in how dsRNA and mRNA responded. The overall trend was that dsRNA labels more effectively with the DNA dye while mRNA labels more effectively with the RNA dye. In the case of the long dsRNA fragment, in all three independent replicates (with two to three repeats each), the area under the curve (FIG. 15B) and peak height (FIG. 15C) were greater using the DNA dye than using the RNA dye. However, while 3/3 of the differences in the area were statistically different, only ⅔ of the peak heights were statistically different. In the case of the long mRNA fragment, the means of each repeat showed greater areas under the curve (FIG. 15E) and peak heights (FIG. 5F) using the RNA dye, however only ⅔ of the area differences were statistically different, and only ⅓ peak heights were statistically different. This suggests that, similar to the previous findings, the area under the curve appears to be a better indicator of difference in response, in part due to the presence of tighter error bars. In the case of the long dsRNA fragment, when comparing the values yielded with the DNA dye to those yielded with the RNA dye, the area saw a decrease of 64% while the peak height saw a decrease of 76%. In the case of the long mRNA fragment, the area saw an increase of 74%, while the peak height saw an increase of 150%.

TABLE 4
Comparison of a long dsRNA fragment peak areas and heights using
different dye types. Experiments were conducted using a DNA dye at
1.2%, an RNA dye at 15.0%, when applicable, and a 3% gel matrix.

| | Area | | Height | |
|---|---|---|---|---|
| | Area DNA | Area RNA dye | Height DNA | Height RNA |
| Run # | dye (F.U.) | (F.U.) | dye (F.U.) | dye (F.U.) |
| 1 | 641.3 ± 4.9 | 220.5 ± 23.5 | 1082.2 ± 124.2 | 359.4 ± 61.1 |
| 2 | 319.4 ± 53.8 | 159.4 ± 9.3 | 376.0 ± 112.7 | 107.8 ± 59.1 |
| 3 | 387.3 ± 103.5 | 112.0 ± 4.0 | 1330.4 ± 526.3 | 192.7 ± 45.5 |
| Average | 449.3 ± 158.1 | 170.48 ± 47.8 | 929.5 ± 510.1 | 223.4 ± 127.6 |

TABLE 5
Comparison of a long mRNA fragment peak areas and heights using
different dye types. Experiments were conducted using a DNA dye at
1.2%, an RNA dye at 15.0%, when applicable, and a 3% gel matrix.

| | Area | | Height | |
|---|---|---|---|---|
| | Area DNA | Area RNA dye | Height DNA | Height RNA |
| Run # | dye (F.U.) | (F.U.) | dye (F.U.) | dye (F.U.) |
| 1 | 4749.1 ± 186.3 | 11399.7 ± 2077.4 | 626.3 ± 42.6 | 2310.0 ± 706.8 |
| 2 | 3220.6 ± 141.7 | 3777.0 ± 632.6 | 375.3 ± 36.7 | 611.4 ± 356.0 |
| 3 | 5082.6 ± 469.0 | 7536.2 ± 576.6 | 622.2 ± 93.9 | 1140.4 ± 119.6 |
| Average | 4350.8 ± 899.0 | 7571.0 ± 3486.8 | 541.3 ± 136.0 | 1354.0 ± 852.5 |

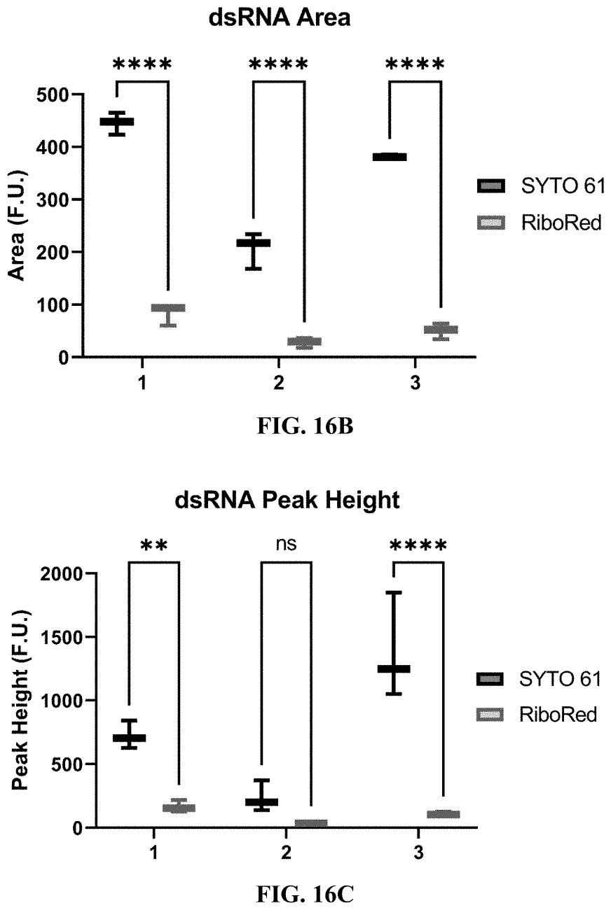

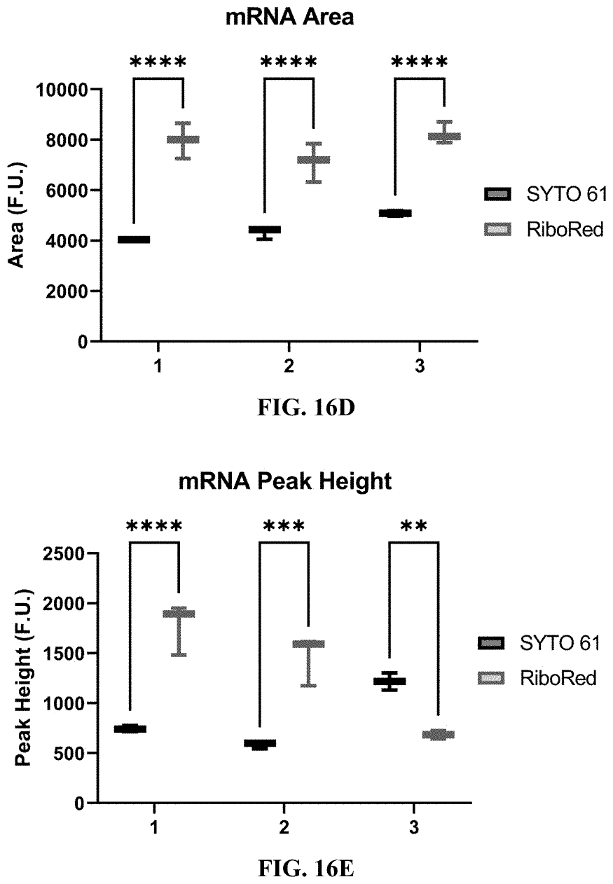

Then, to better simulate a realistic scenario for detecting dsRNA in a mRNA vaccine, we mixed the dsRNA and mRNA samples and assessed their individual response in the presence of both dye types, as seen in FIGS. 16A-16E and Tables 6-7. The dsRNA was used at a concentration of 2.60 ng/μL, while the mRNA was used at a concentration of 5.90/μL based on triplicate nanodrop readings. Similar to experiments with short nucleic acid fragments, dsRNA labeled better with the DNA dye while mRNA labeled better with the RNA dye. In contrast to when the samples were analyzed individually, the dsRNA area decreased by 84% and the peak height decreased by 87% when comparing the values yielded with the DNA dye to those with the RNA dye when the samples were analyzed together. In the case of the mRNA sample, the area showed an increase of 74%, while the peak height saw an increase of 54%. Interestingly, while the changes in the dsRNA sample were further highlighted when the samples were mixed, those in the mRNA sample were less stark. However, regardless of the difference in change when the samples were mixed in comparison to when they were analyzed individually, the statistical difference between the two dyes was higher when the samples were mixed. In the case of the dsRNA, once again, 3/3 areas and ⅔ peak heights were statistically different, while in the case of the mRNA samples, 3/3 areas and 3/3 peak heights were statistically different, whereas before, only ⅔ areas and ⅓ peak heights had shown a statistical difference.

TABLE 6
Comparison of a long dsRNA fragment peak areas and
heights using different dye types when mixed with
mRNA. Experiments were conducted using a DNA dye at 1.2%, an
RNA dye at 15.0%, when applicable, and a 3% gel matrix.

| | Area | | Height | |
|---|---|---|---|---|
| | Area DNA dye | Area RNA dye | Height DNA | Height RNA |
| Run # | (F.U.) | (F.U.) | dye (F.U.) | dye (F.U.) |
| 1 | 445.5 ± 20.8 | 83.6 ± 20.3 | 724.2 ± 108.5 | 166.7 ± 46.2 |
| 2 | 206.4 ± 34.2 | 27.9 ± 9.6 | 237.2 ± 120.9 | 38.6 ± 11.2 |
| 3 | 381.0 ± 4.5 | 50.1 ± 15.1 | 1383.1 ± 415.8 | 110.0 ± 14.3 |
| Average | 344.3 ± 109.0 | 53.9 ± 27.8 | 781.5 ± 545.8 | 105.1 ± 495.0 |

TABLE 7
Comparison of a long mRNA fragment peak areas and heights
using different dye types when mixed with dsRNA. Experiments
were conducted using a DNA dye at 1.2%, an
RNA dye at 15.0%, when applicable, and a 3% gel matrix.

| | Area | | Height | |
|---|---|---|---|---|
| | Area DNA dye | Area RNA dye | Height DNA | Height RNA |
| Run # | (F.U.) | (F.U.) | dye (F.U.) | dye (F.U.) |
| 1 | 4034.8 ± 37.6 | 7971.1 ± 700.0 | 743.6 ± 30.7 | 1775.7 ± 256.5 |
| 2 | 4328.0 ± 237.2 | 7120.1 ± 765.7 | 583.8 ± 35.7 | 1460.1 ± 248.2 |
| 3 | 5082.8 ± 144.5 | 8244.2 ± 425.7 | 1216.6 ± 85.7 | 684.4 ± 59.8 |
| Average | 4405.7 ± 460.4 | 7778.5 ± 756.4 | 848.0 ± 289.2 | 1384.6 ± 495.0 |

While the exact binding methods of the DNA and RNA dye used in this study are not known, our data suggests that the DNA and RNA dye are able to exhibit some selectivity when staining nucleic acids. Without wishing to be bound to the theory, the observed selectivity of the dyes toward different nucleic acids may be due to the different sizes of the dyes and nucleic acids. For example, the RNA dye may have a greater size, making it unable to effectively fit into the grooves or spaces in between dsRNA. Evidence of the lower preference of RNA dye for dsRNA samples can be seen with the dramatic decrease in labeling area of the dsRNA samples when compared to that of the DNA dye. Similarly, the DNA dye may exhibit characteristics that make it too small to effectivity fit and label the ssRNA and mRNA molecules. For example, dsRNA has been known to have an A-form helix, having a length increase per base pair of around 2.8 Åand a radius of around 1.2 nm, causing it to be around 20% shorter and wider than dsDNA, which has a B-form helix in nature [25]. The shorter and wider spacing within dsRNA base pairs and grooves when compared to that of DNA may explain the observation where most fragments in the ssRNA ladder fluoresced brighter than the dsRNA fragments in the initial stages of experimentation with DNA dye. If the DNA dye was created to bind to dsDNA through groove binding or intercalation, it may be too big to bind dsRNA effectively and rather it may fit into the greater spaces in ssRNA.

### Example 6: Detection of dsRNA Contaminants in mRNA Samples Using Peak Areas

This Example describes identification of the type of molecule using the peak areas produced in the presence of the RNA and DNA dyes, which were RiboRed and SYTO™ 61, respectively. We developed a classifier to determine the type of molecule that was yielding a peak given that there was not a statistical difference between the peak heights and even peak areas in some experiments. Based on the behavior observed when mRNA, ssRNA and dsRNA were exposed to different dye molecules, we determined that the type of molecule can be identified based on the following relationship:

$$If : \frac{Peak {Area}_{RNA Dye}}{Peak {Area}_{DNA Dye}} < 1 , then the peak producing molecule is dsRNA . \\ If : \frac{Peak {Area}_{RNA Dye}}{Peak {Area}_{DNA Dye}} > 1 , \\ then the peak producing molecule is mRNA ( or ssRNA ) .$$

TABLE 8
Summarized ratios of the area under the curve of each peak
when labeled with an RNA dye and a DNA dye run in three
independent experiments with two or three repeats.

| | Pure ratio | | Mixed ratio | |
|---|---|---|---|---|
| Run # | dsRNA | mRNA | dsRNA | mRNA |
| 1 | 0.34 ± 0.03 | 2.39 ± 0.34 | 0.19 ± 0.05 | 1.98 ± 0.18 |
| 2 | 0.51 ± 0.11 | 1.17 ± 0.15 | 0.14 ± 0.06 | 1.64 ± 0.11 |
| 3 | 0.34 ± 0.09 | 1.49 ± 0.06 | 0.13 ± 0.04 | 1.66 ± 0.03 |
| Average | 0.40 ± 0.11 | 1.68 ± 0.58 | 0.15 ± 0.05 | 1.77 ± 0.21 |

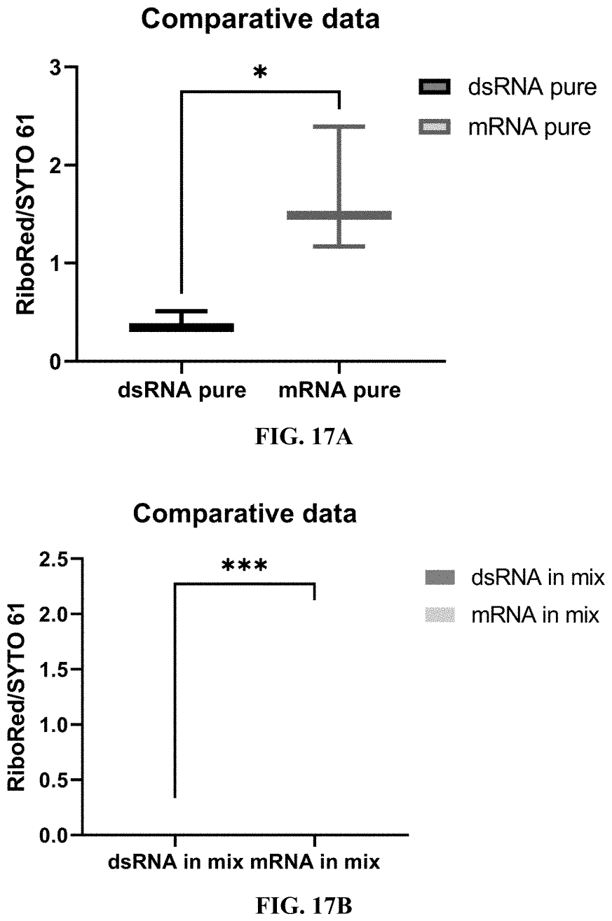

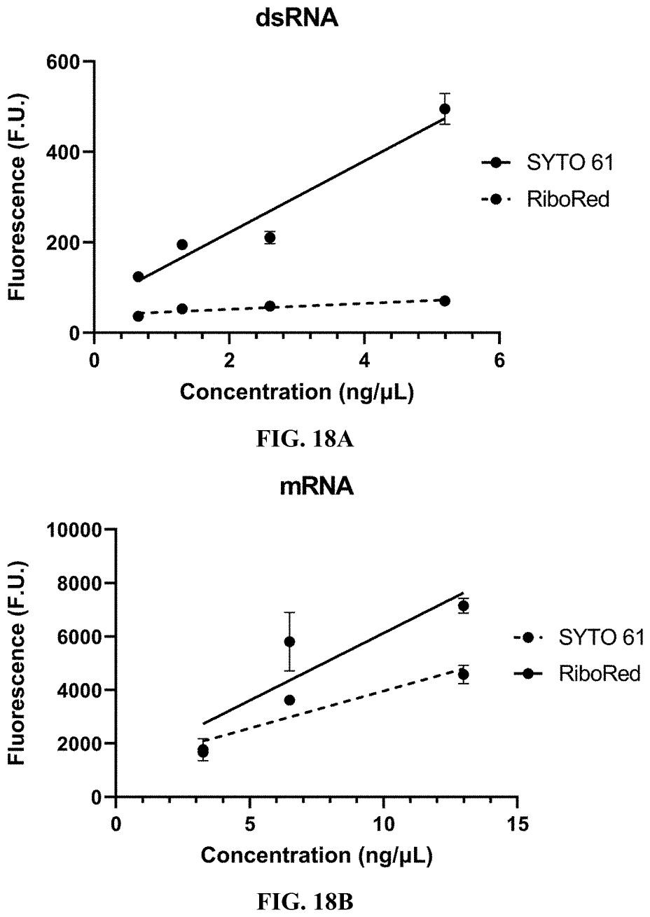

Over all three independent runs (Table 8) the dsRNA peak ratio was <1 while the mRNA ratio was >1. In this case, for each molecule type, 3/3 samples analyzed either in duplicate or triplicate met the differentiation criteria. Interestingly, while the ratios yielded by the mRNA peak in both its pure and mixed form were close in value (1.68 for the pure sample, and 1.77 for the mixed sample), the ratios yielded by the dsRNA were much different (0.40 in its pure form and 0.15 when mixed). This difference is likely due in part to sample degradation that overlaps with the dsRNA peak. For example, in FIG. 16A, there is a baseline increase to the left of the mRNA peak, which is likely due to sample degradation that overlaps with the dsRNA peak. Although the dsRNA and mRNA peaks are overlapping, they are still differentiable. For example, when averaging the three independent runs, there was a statistical difference between the value yielded between the dsRNA sample and the mRNA sample (FIGS. 17A-17B), and therefore the difference between the ratios of the two types of molecules can be determined when they are mixed in a single sample. In addition, the dsRNA fragment used in these experiments was of the same length as the mRNA fragment. This means that shorter dsRNA fragments that may be found in mRNA vaccine samples would migrate faster and overlap even less with the mRNA peak.

### Example 7: Detection of dsRNA Contaminants in mRNA Samples

This Example describes detection of dsRNA contaminants in mRNA samples using methods described herein. The linearity of area to sample concentration was assessed for both types of molecules using the DNA and the RNA dyes, which were SYTO™ 61 and RiboRed, respectively (FIGS. 8A-8B). While all samples showed a roughly linear relation between area and concentration, it was found that for both dsRNA and mRNA, the RNA dye yielded the lowest R² value of 0.75 for both molecules and the DNA dye yielded the highest with values of 0.93 and 0.84, respectively. It was also shown that, for both molecule types, the slope of the line of best fit was highest depending on the dye, specifically the DNA dye in the case of dsRNA and the RNA dye in the case of mRNA. Based on the equations for the lines of best fit yielded for each molecule by both dyes (FIGS. 8A-8B), dsRNA lines would converge when the dsRNA concentration is −0.34 ng/μL while the mRNA lines would converge when the mRNA concentration is 0.38 ng/μL.

Using the areas yielded by each sample from FIGS. 8A-8B and from FIGS. 9A-9B, the limit of detection (L.O.D.) was calculated by determining the minimum concentration that could be observed. This was done by dividing the concentration used by the area yielded. For dsRNA, the areas used were those using the RNA dye since they are lower, and for mRNA, the areas used were those using the DNA dye since they are lower. The results suggested that the L.O.D. for dsRNA is between 17.7-76.6 pg/μL while that of mRNA is between 1.79-2.83 pg/μL. The difference in L.O.D. is consistent with previous experiments showing that, regardless of the dye, both the ssRNA molecules and the mRNA yielded higher areas.

In addition, it is important to note that, while the areas tend to get greater as the concentrations increase, it was determined that the maximum concentration of mRNA that could be used without clogging or affecting the chip was 12.99 ng/μL. Given that we can detect concentrations of dsRNA as low as 17.7-76.6 pg/μL, if we load 12.99 ng/μL of mRNA, we should be able to detect contaminants that are present in percentages as low as 0.14-0.59% in comparison to the total mRNA concentration. Since the actual percentage of dsRNA present in the vaccines has not been thoroughly quantified and characterized, it is of great importance to have a system with a low L.O.D capable of detecting low levels of contaminants.

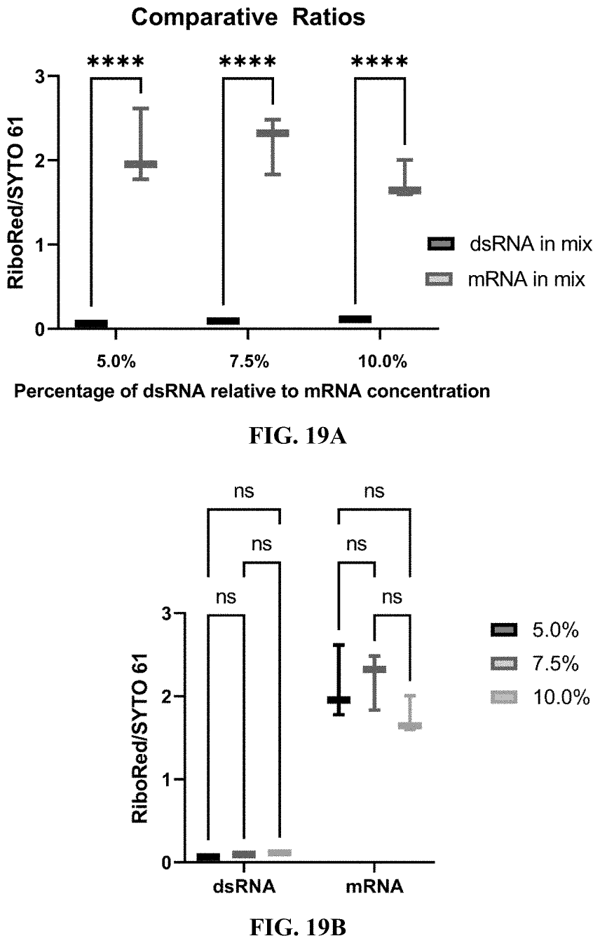

During the synthesis and purification of mRNA vaccines, although mRNA concentrations are regularly controlled, manufacturers have less control over the exact percentage of dsRNA contaminants present in the sample. Therefore, we assessed the ability of the methods described herein to detect various concentrations of dsRNA contaminants relative to the total mRNA concentration. As shown in FIG. 19A, regardless of the dsRNA concentration, there was a difference of equal statistical significance between the dye ratio yielded by the dsRNA peak and that of the mRNA peak. In addition, as shown in FIG. 19B, regardless of the concentration, there was no statistical difference between the ratios yielded by the dsRNA peak at different concentrations, and by the mRNA peak at equal concentration when the dsRNA concentration was modified. These results suggest that, although the area yielded by each peak will vary, the methods described herein are robust and can be useful for detecting dsRNA contaminants regardless of the concentration of dsRNA relative to that of the mRNA in the sample.

### REFERENCES

[1] C. M. C. Rodrigues and S. A. Plotkin, “Impact of Vaccines; Health, Economic and Social Perspectives,” Front Microbiol, vol. 11, p. 1526, July 2020.
[2] J. A. Wolff et al., “Direct gene transfer into mouse muscle in vivo,” Science, vol. 247, no. 4949 Pt 1, pp. 1465-1468, 1990.
[3] A. Wadhwa, A. Aljabbari, A. Lokras, C. Foged, and A. Thakur, “Opportunities and Challenges in the Delivery of mRNA-Based Vaccines,” Pharmaceutics, vol. 12, no. 2, February 2020.
[4] N. Pardi, M. J. Hogan, F. W. Porter, and D. Weissman, “mRNA vaccines—a new era in vaccinology,” Nature Reviews Drug Discovery 2018 17:4, vol. 17, no. 4, pp. 261-279, January 2018.
[5] S. S. Rosa, D. M. F. Prazeres, A. M. Azevedo, and M. P. C. Marques, “mRNA vaccines manufacturing: Challenges and bottlenecks,” Vaccine, vol. 39, no. 16, p. 2190, April 2021.
[6] N. Pardi, M. J. Hogan, and D. Weissman, “Recent advances in mRNA vaccine technology,” Curr Opin Immunol, vol. 65, pp. 14-20, August 2020.
[7] X. Hou, T. Zaks, R. Langer, and Y. Dong, “Lipid nanoparticles for mRNA delivery,” Nature Reviews Materials 2021 6:12, vol. 6, no. 12, pp. 1078-1094, August 2021.
[8] M. Z. Wu, H. Asahara, G. Tzertzinis, and B. Roy, “Synthesis of low immunogenicity RNA with high-temperature in vitro transcription,” RNA, vol. 26, no. 3, pp. 345-360, 2020.
[9] C. de Haro, R. Mendez, and J. Santoyo, “The eIF-2alpha kinases and the control of protein synthesis,” FASEB J, vol. 10, no. 12, pp. 1378-1387, October 1996.
[10] A. K. Minnaert et al., “Strategies for controlling the innate immune activity of conventional and self-amplifying mRNA therapeutics: Getting the message across,” Adv Drug Deliv Rev, vol. 176, p. 113900, September 2021.
[11] N. McGarry et al., “Double stranded RNA drives innate immune responses, sickness behavior and cognitive impairment dependent on dsRNA length, IFNAR1 expression and age.,” bioRxiv, January 2021.
[12] N. McGarry et al., “Double stranded RNA drives anti-viral innate immune responses, sickness behavior and cognitive dysfunction dependent on dsRNA length, IFNAR1 expression and age,” Brain Behav Immun, vol. 95, p. 413, July 2021.
[13] R. Martins, J. A. Queiroz, and F. Sousa, “Ribonucleic acid purification,” J Chromatogr A, vol. 1355, pp. 1-14, August 2014.
[14] K. Karikó, H. Muramatsu, J. Ludwig, and D. Weissman, “Generating the optimal mRNA for therapy: HPLC purification eliminates immune activation and improves translation of nucleoside-modified, protein-encoding mRNA,” Nucleic Acids Res, vol. 39, no. 21, pp. e142-e142, November 2011.
[15] M. Baiersdorfer et al., “A Facile Method for the Removal of dsRNA Contaminant from In Vitro-Transcribed mRNA,” Mol Ther Nucleic Acids, vol. 15, pp. 26-35, April 2019.
[16] Cristina Poveda, A. B. Biter, M. E. Bottazzi, and U. Strych, “Establishing Preferred Product Characterization for the Evaluation of RNA Vaccine Antigens,” Vaccines 2019, Vol. 7, Page 131, vol. 7, no. 4, p. 131, September 2019.
[17] K.-N. Son, Z. Liang, and H. L. Lipton, “Double-Stranded RNA Is Detected by Immunofluorescence Analysis in RNA and DNA Virus Infections, Including Those by Negative-Stranded RNA Viruses,” J Virol, vol. 89, no. 18, p. 9383, September 2015.
[18] C. J. Gabriel, “Detection of double-stranded RNA by immunoblot electrophoresis,” J Virol Methods, vol. 13, no. 4, pp. 279-283,1986.
[19] T. Kartali et al., “Detection and molecular characterization of novel dsrna viruses related to the totiviridae family in Umbelopsis ramanniana,” Front Cell Infect Microbiol, vol. 9, no. JUL, p. 249,2019.
[20] G. M. Whitesides, “The origins and the future of microfluidics,” Nature 2006 442:7101, vol. 442, no. 7101, pp. 368-373, July 2006.
[21] E. K. Sackmann, A. L. Fulton, and D. J. Beebe, “The present and future role of microfluidics in biomedical research,” Nature 2014 507:7491, vol. 507, no. 7491, pp. 181-189, March 2014.
[22] E. K. Sackmann, A. L. Fulton, and D. J. Beebe, “The present and future role of microfluidics in biomedical research,” Nature 2014 507:7491, vol. 507, no. 7491, pp. 181-189, March 2014.
[23] G. M. Whitesides, “The origins and the future of microfluidics,” Nature 2006 442:7101, vol. 442, no. 7101, pp. 368-373, July 2006.
[24] Q. Yao et al., “Differentiating RNA from DNA by a molecular fluorescent probe based on the ‘door-bolt’ mechanism biomaterials,” Biomaterials, vol. 177, pp. 78-87, September 2018.
[25] J. Lipferta et al., “Double-stranded RNA under force and torque: Similarities to and striking differences from double-stranded DNA,” Proc Natl Acad Sci U S A, vol. 111, no. 43, pp. 15408-15413, October 2014.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for separating and detecting double-stranded ribonucleic acids (dsRNAs) from single-stranded ribonucleic acids (ssRNAs) by capillary electrophoresis, the method comprising:
introducing a polymer solution and a sample comprising dsRNAs and ssRNAs into a first capillary channel, wherein the polymer solution is introduced prior to the sample, and wherein the polymer solution or the sample comprises an intercalating agent;
injecting a portion of the sample into a second capillary channel that intersects and is fluidly connected with the first capillary channel;
applying a voltage gradient across the length of the second capillary channel to separate dsRNAs into a first segment of the portion of the sample and ssRNAs into a second segment of the portion of the sample; and
detecting an amount of intercalating agent in the first segment and the second segment, wherein the amount of intercalating agent in the first segment is indicative of an amount of dsRNA in the sample and the amount of intercalating agent in the second segment is indicative of an amount of ssRNA in the sample.

2. The method of claim 1, wherein the intercalating agent is fluorescent and detecting the amount of intercalating agent comprises detecting a fluorescent signal.

3. (canceled)

4. The method of claim 1, wherein the intercalating agent is selected from the group consisting of SYTO™ dyes, Ribo dyes, SYBR dyes, ethidium bromide, acridine orange, propidium iodide, 7-aminoactinomycin D (7-AAD), 4′,6-diamidino-2-phenylindole (DAPI), cyanine dyes, Alexa Fluor dyes, BODIPY dyes, and combinations thereof.

5.-6. (canceled)

7. The method of claim 1, wherein the polymer is formed from a monomer selected from the group consisting of acrylic acid, carboxylic acid, sulfonic acid, phosphoric acid monomer, bisacrylamidoacetic acid, 4,4-Bis(4-hydroxyphenyl)pentanoic acid, 3-butene-1,2,3-tricarboxylic acid, 2-carboxyethylacrylate, itaconic acid, methacrylic acid, and 4-vinylbenzoic acid.

8. The method of claim 1, wherein the polymer is an acrylic polymer.

9. The method of claim 8, wherein the acrylic polymer is selected from the group consisting of a polyacrylamide polymer, a polymethylacrylamide polymer, a polydimethylacrylamide polymer, and a polydimethylacrylamide-co-acrylic acid polymer.

10. The method of claim 1, wherein the polymer comprises a net charge of between 0.01% and 2% and the net charge is the same charge as at least one surface of the capillary channel.

11. The method of claim 10, wherein the polymer comprises negatively charged monomer subunits and the at least one surface of the capillary channel has a negative surface charge.

12. The method of claim 11, wherein the negatively charged monomer subunits are selected from the group consisting of are selected from acrylic acid, bisacrylamidoacetic acid, 4,4-bis(4-hydroxyphenyl)pentanoic acid, 3-butene-1,2,3-tricarboxylic acid, 2-carboxyethylacrylate, itaconic acid, methacrylic acid, 4-vinylbenzoic acid, sulfonic acid, 2-acrylamido-2-methyl-l-propanesulfonic acid, 2-methyl-2-propene-l-sulfonic acid, 2-propene-1-sulfonic acid, 4-styrenesulfonic acid, 2-sulfoethyl methacrylate, 3-sulfopropyldimethyl-3-methacrylamidopropylammonium inner salt, 3-sulfopropyl methacrylate, vinylsulfonic acid, bis(2-methacryloxyethyl)phosphate, and monoacryloxyethyl phosphate.

13. The method of claim 10, wherein the polymer comprises positively charged monomer subunits and the at least one surface of the capillary channel has a positive surface charge.

14. The method of claim 13, wherein the positively charged monomer subunits are selected from the group consisting of 2-acryloxyethyltrimethylammonium chloride, diallyldimethylammonium chloride, 2-methacryloxyethyltrimethylammonium chloride, and 3-methacryloxy-2-hydroxypropyltrimethylammonium chloride.

15.-16. (canceled)

17. The method of claim 1, wherein injecting the portion of the sample from the capillary channel into the second capillary channel comprises applying a voltage difference through the capillary channel to electrokinetically move the portion of the sample from the capillary channel into the second capillary channel.

18. The method of claim 1, further comprising:
introducing an additional polymer solution and the sample into the first capillary channel, wherein the additional polymer solution is introduced prior to the sample, and wherein the additional polymer solution or the sample comprises an additional intercalating agent;
injecting a portion of the sample into the second capillary channel that intersects and is fluidly connected with the first capillary channel;
applying a second voltage gradient across the length of the second capillary channel to separate dsRNAs into an additional first segment of the portion of the sample and ssRNAs into an additional second segment of the portion of the sample; and
detecting an amount of additional intercalating agent in the additional first segment and the additional second segment, wherein the amount of additional intercalating agent in the additional first segment is indicative of an amount of dsRNA in the sample and the amount of additional intercalating agent in the additional second segment is indicative of an amount of ssRNA in the sample.

19. (canceled)

20. A method for separating and detecting double-stranded ribonucleic acids (dsRNAs) from single-stranded ribonucleic acids (ssRNAs) in a microfluidic device, the method comprising:
providing a microfluidic device comprising:
a substrate having a surface;
an analysis channel disposed in the substrate;
a sample loading channel disposed in the substrate and intersecting the analysis channel at an intersection; and
introducing a polymer solution and a sample comprising dsRNAs and ssRNAs into the loading channel, wherein the polymer solution is introduced prior to the sample, and wherein the polymer solution or the sample comprises an intercalating agent;
injecting a portion of the sample into the analysis channel;
applying a voltage gradient across the length of the analysis channel to separate dsRNAs into a first segment of the portion of the sample and ssRNAs into a second segment of the portion of the sample; and
detecting an amount of intercalating agent in the first segment and the second segment, wherein the amount of intercalating agent in the first segment is indicative of an amount of dsRNA in the sample and the amount of intercalating agent in the second segment is indicative of an amount of ssRNA in the sample.

21. The method of claim 20, wherein the intercalating agent is fluorescent and the detecting step comprises detecting a fluorescent signal.

22. (canceled)

23. The method of claim 20, wherein the intercalating agent is selected from the group consisting of SYTO™ dyes, Ribo dyes, SYBR dyes, ethidium bromide, acridine orange, propidium iodide, 7-aminoactinomycin D (7-AAD), 4′,6-diamidino-2-phenylindole (DAPI), cyanine dyes, Alexa Fluor dyes, BODIPY dyes, and combinations thereof.

24.-25. (canceled)

26. The method of claim 20, wherein the polymer is formed from a monomer selected from the group consisting of acrylic acid, carboxylic acid, sulfonic acid, phosphoric acid monomer, bisacrylamidoacetic acid, 4,4-bis(4-hydroxyphenyl)pentanoic acid, 3-butene-1,2,3-tricarboxylic acid, 2-carboxyethylacrylate,itaconic acid, methacrylic acid, and 4-vinylbenzoic acid.

27. The method of claim 20, wherein the polymer is an acrylic polymer.

28. The method of claim 27, wherein the acrylic polymer is selected from the group consisting of a polyacrylamide polymer, a polymethylacrylamide polymer, a polydimethylacrylamide polymer, and a polydimethylacrylamide-co-acrylic acid polymer.

29.-35. (canceled)

36. The method of claim 20, further comprising:
introducing an additional polymer solution and the sample into the first capillary channel, wherein the additional polymer solution is introduced prior to the sample, and wherein the additional polymer solution or the sample comprises an additional intercalating agent;
injecting a portion of the sample into the second capillary channel that intersects and is fluidly connected with the first capillary channel;
applying a second voltage gradient across the length of the second capillary channel to separate dsRNAs into an additional first segment of the portion of the sample and ssRNAs into an additional second segment of the portion of the sample; and
detecting an amount of additional intercalating agent in the additional first segment and the additional second segment, wherein the amount of additional intercalating agent in the additional first segment is indicative of an amount of dsRNA in the sample and the amount of additional intercalating agent in the additional second segment is indicative of an amount of ssRNA in the sample.
